(19) United States
(12) Patent Application Publication
(10) Pub. No.: US 2026/0315476 A1
(43) Pub. Date: Oct. 15, 2026

(54) COMBINATION TREATMENT METHODS FOR CANCER WITH TP53 MUTATION

(71) Applicant: HO'OLA THERAPEUTICS INC., Belmont, CA (US)

(72) Inventors: Salim YAZJI, San Diego, CA (US); James BREITMEYER, San Diego, CA (US); Gunnar KAUFMANN, San Diego, CA (US)

(21) Appl. No.: 19/104,172

(22) PCT Filed: Jun. 13, 2023
(86) PCT No.: PCT/US2023/068368

§ 371 Date: Feb. 14, 2025

Related U.S. Application Data
(63) Continuation-in-part of application No. PCT/US2022/040879, filed on Aug. 19, 2022.
(60) Provisional application No. 63/487,193, filed on Feb. 27, 2023.

(51) Int. Cl. A61K 31/519; A61K 39/00; A61K 39/395; A61P 35/00; C07K 16/28

(52) U.S. Cl. CPC A61K 31/519; A61K 39/3955; A61P 35/00; C07K 16/2803; A61K 2039/505

(57) ABSTRACT

What is disclosed herein is a method for treating a cancer or tumor in an individual, comprising administering to the individual an ROR1 antagonist and a Bruton's tyrosine kinase inhibitor (BTKi), wherein the tumor or cancer comprises a mutation in a TP53 gene. The method may halt progression of the cancer for at least 12 months as compared to an individual that does not comprise a mutation in a TP53 gene.

### CROSS REFERENCE TO RELATED APPLICATIONS

This application claims the benefit of International Application No. PCT/US2022/040879 filed Aug. 19, 2022, and U.S. Provisional Application No. 63/487,193 filed Feb. 27, 2023, each of which are incorporated herein by reference in their entirety.

### INCORPORATION BY REFERENCE OF SEQUENCE LISTING

The present application is being filed along with a Sequence Listing in electronic format. The Sequence Listing is provided as a file entitled 51956-727-602_SEQ.xml, created Jun. 12, 2023, which is 12,620 bytes in size. The information in the electronic format of the Sequence Listing is incorporated by reference in its entirety.

### BACKGROUND OF THE INVENTION

Receptor-tyrosine kinase like orphan receptor 1 (ROR1) is a conserved embryonic protein whose expression becomes progressively reduced during embryonic development in mammals. However, ROR1 is found to be re-expressed at high levels on many solid and hematologic cancers including mantle cell lymphoma (MCL), chronic lymphocytic leukemia (CLL), and marginal zone lymphoma (MZL), but not on normal adult tissues. Although there are many therapies available for treating these diseases, these diseases are incurable and there remain a need for more effective treatment, especially for certain patients, such as those with cancer or tumor comprising TP53 mutations.

### SUMMARY OF THE INVENTION

It is appreciated by the inventors that the treatment of various solid and hematologic cancers may be improved by targeting patient populations likely to respond to a particular therapy. Disclosed herein is a method of treating a cancer or tumor in an individual, comprising administering to the individual an ROR1 antagonist and/or a Bruton's tyrosine kinase inhibitor (BTKi), wherein the tumor or cancer comprises a mutation in a TP53 gene or is a high-risk subtype. In certain embodiments, the ROR1 antagonist is an antibody or antigen binding fragment thereof. In certain embodiments, the antibody or antigen binding fragment thereof binds to an epitope of ROR1 as set forth in SEQ ID NO: 13 or 14. In certain embodiments, the antibody or antigen biding fragment thereof comprises: a) a heavy chain CDR1 (HCDR1) comprising the amino acid sequence set forth in SEQ ID NO: 1; b) a heavy chain CDR2 (HCDR2) comprising the amino acid sequence set forth in SEQ ID NO: 2; c) a heavy chain CDR3 (HCDR3) comprising the amino acid sequence set forth in SEQ ID NO: 3; d) a light chain CDR1 (LCDR1) comprising the amino acid sequence set forth in SEQ ID NO: 4; e) a light chain CDR2 (LCDR2) comprising the amino acid sequence set forth in SEQ ID NO: 5; and/or f) a light chain CDR3 (LCDR3) comprising the amino acid sequence set forth in SEQ ID NO: 6. In certain embodiments, the antibody or antigen biding fragment thereof comprises a heavy chain variable region comprising an amino acid sequence at least about 85% identical to that set forth in SEQ ID NO: 7; and/or a light chain variable region comprising an amino acid sequence at least about 85% identical to that set forth in SEQ ID NO: 8. In certain embodiments, the antibody or antigen biding fragment thereof is zilovertamab or an ROR1 binding fragment thereof. In certain embodiments, the ROR1 antagonist is administered at a dose of between 200 milligrams and 800 milligrams. In certain embodiments, the ROR1 antagonist is administered at a dose of 200 milligrams. In certain embodiments, the ROR1 antagonist is administered at a dose of 400 milligrams. In certain embodiments, the ROR1 antagonist is administered at a dose of 600 milligrams. In certain embodiments, the BTKi comprises ibrutinib, acalabrutinib, evobrutinib, fenebrutinib, orelabrutinib, rilzabrutinib, or tolebrutinib. In certain embodiments, the BTKi comprises branebrutinib, elsubrutinib, remibrutinib, vecabrutinib, remibrutinib, spebrutinib tirabrutinib, AC0058TA, LOXO-305, DTRMWXHS-12, M7583, SN-1011, TG-1701. In certain embodiments, the BTKi comprises ibrutinib. In certain embodiments, the BTKi comprises nemtabrutinib. In certain embodiments, the BTKi is administered at a dose of between about 280 and about 700 milligrams. In certain embodiments, the BTKi is administered at a dose of about 420 milligrams. In certain embodiments, the BTKi is administered at a dose of about 560 milligrams. In certain embodiments, the ROR1 antagonist and the BTKi are administered separately. In certain embodiments, the tumor or the cancer comprises a blood cancer. In certain embodiments, the tumor or the cancer comprises a leukemia or a lymphoma. In certain embodiments, the tumor or the cancer comprises a B cell leukemia or B cell lymphoma. In certain embodiments, the tumor or the cancer comprises chronic lymphocytic leukemia (CLL). In certain embodiments, the tumor or the cancer comprises small cell lymphocytic leukemia (SLL). In certain embodiments, the tumor or the cancer comprises marginal zone lymphoma (MZL). In certain embodiments, the cancer or tumor is relapsed. In certain embodiments, the cancer or refractory to one or more previous treatments. In certain embodiments, the MCL is a high-risk subtype according to the simplified mantle cell lymphoma international prognostic index. In certain embodiments, the mutation is in an exon of the TP53 gene. In certain embodiments, the mutation is in an intron of the TP53 gene. In certain embodiments, the mutation is a nonsense mutation in the TP53 gene. In certain embodiments, the mutation is a missense mutation in the TP53 gene. In certain embodiments, the mutation results in an amino acid insertion, deletion, or substitution in a protein encoded by the TP53 gene. In certain embodiments, the cancer or tumor is relapsed. In certain embodiments, the cancer or tumor is refractory to one or more previous treatments. In certain embodiments, the test is performed on a biopsy sample of the tumor or the cancer of the individual. In certain embodiments, the method halts progression of the cancer. In certain embodiments, the method halts progression of the cancer for at least 12 months. In certain embodiments, the method halts progression of the cancer at least 24 months. In certain embodiments, the method halts progression of the cancer for at least 36 months. In certain embodiments, the method halts progression of the cancer for at least 48 months.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D illustrate a waterfall plot of best percentage (%) tumor reduction from baseline. FIG. 1A shows the percentage tumor reduction in MCL patients (Parts 1&2, n=27) treated with zilovertamab and a Bruton's Kinase Inhibitor BTKI (e.g., ibrutinib). FIG. 1B shows the percentage tumor reduction in CLL patients (Parts 1&2, n=34) treated with zilovertamab and a Bruton's Kinase Inhibitor BTKI (e.g., ibrutinib). The CLL patients were either treatment-naïve or relapsed/refractory. FIG. 1C shows the percentage tumor reduction in CLL patients (Part 3, n=15^{a}) treated with zilovertamab and a Bruton's Kinase Inhibitor BTKI (e.g., ibrutinib). The CLL patients were either treatment-naïve or relapsed/refractory. ^{a}: tumor measurements not available for 1 subject in zilovertamab+ibrutinib arm. FIG. 1D shows the percentage tumor reduction in CLL patients (Part 3, n=7) treated with a Bruton's Kinase Inhibitor BTKI (e.g., ibrutinib) alone as a control group. The CLL patients were either treatment-naïve or relapsed/refractory. SPD: sum of the products of the diameters. The number under the bars represent baseline SPD.

FIGS. 2A-2B illustrate the comparison of clinical response rates between the combination treatment (zilovertamab+ibrutinib) and a Bruton's Kinase Inhibitor BTKI (e.g., ibrutinib) treatment based on historical data. FIG. 2A shows the clinical response rates over time of the combination treatment (zilovertamab+ibrutinib). FIG. 2B shows the clinical response rates over time of a Bruton's Kinase Inhibitor BTKI (e.g., ibrutinib) treatment alone.

FIGS. 3A-3B illustrate the comparison of clinical response rates based on prior regiment in MCL patients between the combination treatment (zilovertamab+ibrutinib) and ibrutinib treatment based on historical data. FIG. 3A shows the clinical response rates of the combination treatment (zilovertamab+ibrutinib). FIG. 3B shows the clinical response rates of ibrutinib treatment alone.

FIG. 4 illustrates the comparison of clinical response rates based on p53 mutation status in MCL patients treated with the combination treatment (zilovertamab+ibrutinib).

FIGS. 5A-5B illustrate the comparison of Progression-free Survival (PFS) in MCL patients (Parts 1&2) between the combination treatment (zilovertamab+ibrutinib) and a Bruton's Kinase Inhibitor BTKI (e.g., ibrutinib) treatment based on historical data. FIG. 5A shows the PFS of the combination treatment (zilovertamab+ibrutinib). FIG. 5B shows the PFS of a Bruton's Kinase Inhibitor BTKI (e.g., ibrutinib) treatment alone. PFS is defined as the time from the first dose to the time of disease progression of death from any cause, whichever comes first; NE=Not evaluable; ^{a}-Patient-level data from three single-agent ibrutinib studies, N=370 from Rule, Br J Haematol., 2017.

FIGS. 6A-6B illustrate the comparison of PFS in MCL patients (Parts 1&2) based on prior systemic treatment between the combination treatment (zilovertamab+ibrutinib) and ibrutinib treatment based on historical data. FIG. 6A shows the PFS of the combination treatment (zilovertamab+ibrutinib). FIG. 6B shows the PFS of a Bruton's Kinase Inhibitor BTKI (e.g., ibrutinib) treatment alone. PFS is defined as the time from the first dose to the time of disease progression of death from any cause, whichever comes first; ^{a}-Patient-level data from three single-agent ibrutinib studies, N=370 from Dreyling, HemaSphere, 2022.

FIGS. 7A-7B illustrate the comparison of PFS in MCL patients (Parts 1&2) based on MIPI subgroups between the combination treatment (zilovertamab+ibrutinib) and a Bruton's Kinase Inhibitor BTKI (e.g., ibrutinib) treatment based on historical data. FIG. 7A shows the PFS of the combination treatment (zilovertamab+ibrutinib). FIG. 7B shows the PFS of a Bruton's Kinase Inhibitor BTKI (e.g., ibrutinib) treatment alone. PFS is defined as the time from the first dose to the time of disease progression of death from any cause, whichever comes first; ^{a}-subgroups by sMIPI: Simplified Mantle Cell Lymphoma International Prognostic Index; ^{b}-subgroups by MIPI: Mantle Cell Lymphoma International Prognostic Index; Patient-level data from three single-agent studies, N=370 from Rule, Br J Haematol., 2017.

FIG. 8 illustrates the comparison of PFS in MCL patients (Parts 1&2) based on p53 mutation status between the combination treatment (zilovertamab+ibrutinib) and a Bruton's Kinase Inhibitor BTKI (e.g., ibrutinib) treatment based on historical data. PFS is defined as the time from the first dose to the time of disease progression of death from any cause, whichever comes first; NE=not evaluable; NR=not reached; ^{a}-Rule, Hematologica, 2019.

FIGS. 9A-9C illustrate the comparison of PFS in treatment naïve and relapsed/refractory CLL patients (Parts 1&2, Part 3). FIG. 9A shows PFS comparison in CLL patients (Parts 1 & 2).

FIG. 9B shows PFS comparison in CLL patients (Part 3). FIG. 9C shows the comparison of PFS in treatment naïve and relapsed/refractory CLL patients (pooled analysis of Parts 1, 2 & 3).

FIGS. 10A-10C illustrate the comparison of PFS in CLL patients by p53 mutation status. FIG. 10A shows PFS comparison in CLL patients (Parts 1 & 2). FIG. 10B shows PFS comparison in pooled CLL patients (Parts 1, 2 & 3). PFS is defined as the time from the first dose to the time of disease progression of death from any cause, whichever comes first; ^{a}-Pooled analysis includes all Part 1 and 2 CLL patients+Part 3 CLL patients randomized to treatment with zilovertamab+ibrutinib. FIG. 10C shows PFS comparison in pooled CLL patients (Parts 1, 2 & 3) and compares it to the PFS in subjects with a p53 mutation treated with either ibrutinib or zanuburtinib alone.

FIG. 11 illustrates PFS in CLL patients by prior systemic treatment. PFS is defined as the time from the first dose to the time of disease progression of death from any cause, whichever comes first, through 36 months; NR=not reached; NE=not estimable; ^{a}-min, max used when confidence interval is not estimable.

FIGS. 12A-12B illustrate the comparison of Landmark PFS in R/R CLL patients (Parts 1&2) by prior systemic treatment between the combination treatment (zilovertamab+ibrutinib) and ibrutinib treatment based on historical data. FIG. 12A shows the Landmark PFS of the combination treatment (zilovertamab+ibrutinib). FIG. 12B shows the PFS of a Bruton's Kinase Inhibitor BTKI (e.g., ibrutinib) treatment alone. PFS is defined as the time from the first dose to the time of disease progression of death from any cause, whichever comes first; NR=not reached; ^{a}-PFS by prior therapies from sing-agent a Bruton's Kinase Inhibitor BTKI (e.g., ibrutinib) treatment in high-risk, relapsed patients with CLL (N=195), Byrd, Blood 2019.

### DETAILED DESCRIPTION OF THE INVENTION

Disclosed herein is a method of treating a cancer or tumor in an individual, comprising administering to the individual an-ROR1 antagonist wherein the tumor or cancer comprises a mutation in a TP53 gene or a high-risk subtype. In certain embodiments, the individual has been administered or is being concurrently administered a Bruton's tyrosine kinase inhibitor (BTKi).

Disclosed herein is a method of treating a cancer or tumor in an individual, comprising administering to the individual a Bruton's tyrosine kinase inhibitor (BTKi), wherein the tumor or cancer comprises a mutation in a TP53 gene or a high-risk subtype. In certain embodiments, the individual has been administered or is being concurrently administered an ROR1 antagonist.

Disclosed herein is a method of treating a cancer or tumor in an individual, comprising administering to the individual an-ROR1 antagonist and/or a Bruton's tyrosine kinase inhibitor (BTKi), wherein the tumor or cancer comprises a mutation in a TP53 gene or a high-risk subtype.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. In some instances, “about” means a range extending to +/−10% of the specified value.

The term “Bruton's tyrosine kinase,” also known as tyrosine-protein kinase BTK, as used herein refers to the any of the recombinant or naturally-occurring forms of Bruton's tyrosine kinase (BTK) or variants or homologs thereof that maintain BTK activity (e.g., within at least 50%, 80%, 90%, 95%, 96%, 97%, 98%, 99% or 100% activity compared to BTK). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring BTK protein. In some instances, the BTK protein is substantially identical to the protein identified by the UniProt reference number Q01687 or a variant or homolog having substantial identity thereto.

The term “BTK inhibitor” or “BTKi” as provided herein refers to a substance capable of inhibiting BTK activity compared to a control. The inhibited activity of BTK can be 10%, 20%, 30%, 40%, 50%, 60%, 70%, 80%, 90% or less than that in a control. In some instances, the inhibition is 1.5-fold, 2-fold, 3-fold, 4-fold, 5-fold, 10-fold, or more in comparison to a control. A BTKi inhibits BTK activity e.g., by at least in part, partially or totally blocking stimulation, decreasing, preventing, or delaying activation, or inactivating, desensitizing, or down-regulating signal transduction, activity or amount of BTK relative to the absence of the BTKi.

The term “ROR1” as used herein refers to the any of the recombinant or naturally-occurring forms of tyrosine kinase-like orphan receptor 1 (ROR1) or variants or homologs thereof that maintain ROR1 activity (e.g., within at least 50%, 80%, 90%, 95%, 96%, 97%, 98%, 99% or 100% activity compared to ROR1). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring ROR1 protein. In some instances, the ROR1 protein is substantially identical to the protein identified by Accession No. NP_005003.1 or a variant or homolog having substantial identity thereto. In some instances, the ROR1 protein comprises the amino acid sequence of SEQ ID NO: 12. In some instances, the ROR1 protein consists of the amino acid sequence of SEQ ID NO: 12. In some instances, the ROR1 protein comprises or consists of the amino acid sequence of SEQ ID NO:13. In some instances, the ROR1 protein comprises or consists of the amino acid sequence of SEQ ID NO: 14.

The term “ROR1 antagonist” as provided herein refers to a substance capable of inhibiting ROR1 activity compared to a control. The inhibited activity of ROR1 can be 10%, 20%, 30%, 40%, 50%, 60%, 70%, 80%, 90% or less than that in a control. In some instances, the inhibition is 1.5-fold, 2-fold, 3-fold, 4-fold, 5-fold, 10-fold, or more in comparison to a control. A ROR1 antagonist inhibits ROR1 activity e.g., by at least in part, partially or totally blocking stimulation, decreasing, preventing, or delaying activation, or inactivating, desensitizing, or down-regulating signal transduction, activity or amount of ROR1 relative to the absence of the ROR1 antagonist. In some instances, the ROR1 antagonist is an antibody or small molecule. The term “antagonist” may alternatively be used herein as inhibitor.

The terms “tumor protein p53” and “p53” used interchangeably herein, refer to a potent tumor suppressor protein. “TP53” refers to the gene encoding the tumor suppressor protein. TP53 is negatively regulated or mutated in many cancers. Absence or inactivation of TP53 may contribute to cancer. A wide variety of TP53 mutations exist. In some cases, a cancer may overexpress p53, in particular, mutant versions of p53. A “wild type” TP53 is TP53 found in normal (i.e., non-cancerous cells) or TP53 that does not have a mutation correlated to a cancer. The TP53 status of a sample (e.g., whether the sample includes wild-type or mutant TP53) may be assessed as for example described in U.S. Pat. No. 6,090,566 issued to Vogelstein et al., or using standard techniques such as described herein or known in the art. A p53 protein may include, without limitation, polypeptides containing sequences substantially identical to that set forth in for example UniProt Accession No. P04637. A TP53 molecule may include, without limitation, nucleic acid molecules encoding polypeptides containing sequences substantially identical to that set forth in for example UniProt Accession No. P04637. In some instances, the amino acid sequence of p53 is the sequence of SEQ ID NO: 11. In some instances, p53 comprises an amino acid sequence of SEQ ID NO: 11.

The term “mutation” as referred to herein refers to any genetic alteration that compromises wildtype function or expression of an mRNA, protein, or polypeptide encoded by the gene. Such alterations include missense or non-sense mutations, gene deletions or translocations, insertion of one or more nucleic acids and the like. Mutations are not limited to exon sequences and can occur in introns, 3′ or 5′ untranslated regions, regulatory regions (including promoters and enhancers) of genes.

As described herein an “epitope” refers to the binding determinant of an antibody or fragment described herein minimally necessary for specific binding of the antibody or fragment thereof to a target antigen. When the target antigen is a polypeptide the epitope will be a continuous or discontinuous epitope. A continuous epitope is formed by one region of the target antigen, while a discontinuous epitope may be formed from two or more separate regions. A discontinuous epitope, for example, may form when a target antigen adopts a tertiary structure that brings two amino acid sequences together and forms a three-dimensional structure bound by the antibody. When the target antigen is a polypeptide the epitope will generally be a plurality of amino acids linked into a polypeptide chain. A continuous epitope may comprise 5, 6, 7, 8, 9, 10, 11, 12, 13, 14, 15, 16, 17, 18, 19, or 20 contiguous amino acids. While an epitope may comprise a contiguous polymer of amino acids, not every amino acid of the polymer may be contacted by an amino acid residue of the antibody. Such non-contacted amino acids will still comprise part of the epitope as they may be important for the structure and linkage of the contacted amino acids. The skilled artisan may determine if any given antibody binds an epitope of a reference antibody, for example, by cross-blocking experiments with a reference antibody. In certain embodiments, described herein, are antibodies that bind the same epitope of the described antibodies. In certain embodiments, described herein, are antibodies that are competitively blocked by the described antibodies. In certain embodiments, described herein, are antibodies that compete for binding with the described antibodies.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies (e.g., scFv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies of this disclosure. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

For preparation of suitable antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies of this disclosure. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well-known procedures from a variety of human cells.

The terms “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., ROR1) in other proteins with different numbering systems. For example, by performing a simple sequence alignment with a protein (e.g., ROR1), the identity and location of residues corresponding to specific positions of said protein are identified in other protein sequences aligning to said protein. For example, a selected residue in a selected protein corresponds to glutamic acid at position 138 when the selected residue occupies the same essential spatial or other structural relationship as a glutamic acid at position 138. In some instances, where a selected protein is aligned for maximum homology with a protein, the position in the aligned selected protein aligning with glutamic acid 138 is said to correspond to glutamic acid 138. Instead of a primary sequence alignment, a three-dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is said to correspond to the glutamic acid 138 residue.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

By “therapeutically effective dose or amount” as used herein is meant a dose that produces effects for which it is administered (e.g., treating or preventing a disease). The exact dose and formulation will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The term “ibrutinib,” also known as Imbruvica®, PCI 32765 or the like, refers in the usual and customary sense, to 1-[(3R)-3[4-Amino-3-(4-phenoxyphenyl)-1H-pyrazolo[3,4-d]pyrimidin-1-yl]piperidin-1-yl]prop-2-en-1-one (CAS Registry number 936563-96-1). In some instances, the BTK inhibitor is any one of the compounds disclosed in U.S. Pat. Nos. 7,514,444; 8,008,309; 8,497,277; 8,476,284; 8,697,711, and 8,703,780 which are incorporated by reference herein in their entirety and for all purposes.

The term “idelalisib,” also known as CAL101, GS-1101, Zydelig® or the like, refers in the usual and customary sense to 5-Fluoro-3-phenyl-2-[(1S)-1-(7H-purin-6-ylamino) propyl]-4(3H)-quinazolinone (CAS Registry number 870281-82-6). In some instances, the BTK inhibitor is any one of the compounds disclosed in U.S. Pat. Nos. 9,469,643; 9,492,449; 8,139,195; 8,492,389; 8,865,730; and 9,149,477 which are incorporated by reference herein in their entirety and for all purposes.

The term “R406” or the like refers, in the usual and customary sense, to 6-((5-fluoro-2-((3,4,5-trimethoxyphenyl)amino)pyrimidin-4-yl)amino)-2,2-dimethyl-2H-pyrido[3,2-b][1,4]oxazin-3 (4H)-one benzenesulfonate.

The term “fostamatinib” or the like refers in the usual and customary sense, to 6-((5-fluoro-2-((3,4,5-trimethoxyphenyl)amino)pyrimidin-4-yl)amino)-2,2-dimethyl-2H-pyrido[3,2-b][1,4]oxazin-3 (4H)-one benzenesulfonate (CAS Registry number 901119-35-5 or 1025687-58-4 (disodium salt)). Fostamatinib is a prodrug of R406. In some instances, the BTK inhibitor is any one of the compounds disclosed in U.S. Pat. No. 7,449,458 which is incorporated by reference herein in its entirety and for all purposes.

The term “acalabrutinib,” also known as ACP-196 or the like, refers in the usual and customary sense to 4-[8-amino-3-[(2S)-1-but-2-ynoylpyrrolidin-2-yl]imidazo[1,5-a]pyrazin-1-yl]-N-pyridin-2-ylbenzamide (CAS Registry number 1420477-60-6). In some instances, the BTK inhibitor is any one of the compounds disclosed in U.S. patents application Nos. 20140155385, 20160151364, 20160159810 which are incorporated by reference herein in their entirety and for all purposes.

The term “ONO/GS-4059” also known as tirabrutinib or the like, refers in the usual and customary sense to 6-amino-7-(4-phenoxyphenyl)-9-[(3S)-1-prop-2-enoylpiperidin-3-yl]purin-8-one (CAS Registry number 1351636-18-4). In some instances, the BTK inhibitor is any one of the compounds disclosed in U.S. Pat. Nos. 8,940,725 and 8,557,803 and US Patent Application No. 20150094299 which are incorporated by reference herein in their entirety and for all purposes.

The term “BGB-3111” also known as zanubrutinib and the like, refers in the usual and customary sense to 2-(4-phenoxyphenyl)-7-(1-prop-2-enoylpiperidin-4-yl)-1,5,6,7-tetrahydropyrazolo[1,5-a]pyrimidine-3-carboxamide (CAS Registry number 1633350-06-7). In some instances, the BTK inhibitor is any one of the compounds disclosed in U.S. Pat. Nos. 9,447,106 and 9,556,188 which are incorporated by reference herein in their entirety and for all purposes.

The term “CC-292,” also known as AVL-292, spebrutinib or the like, refers in the usual and customary sense to N-[3-[[5-fluoro-2-[4-(2-methoxyethoxy)anilino]pyrimidin-4-yl]amino]phenyl]prop-2-enamide (CAS Registry number 1202757-89-8). In some instances, the BTK inhibitor is any one of the compounds disclosed in U.S. Pat. No. 8,338,439 which is incorporated by reference herein in its entirety and for all purposes.

The terms “zilovertamab”, “cirmtuzumab”, “UC-961”, and “99961.1” refer to a humanized monoclonal antibody capable of binding the extracellular domain of the human receptor tyrosine kinase-like orphan receptor 1 (ROR1). In some instances, cirmtuzumab is any one of the antibodies or fragments thereof disclosed in U.S. Pat. No. 9,758,591, which is incorporated by reference herein in its entirety and for all purposes.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, aleukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some instances, a patient is human.

The term “relapse”, as used herein, refers to the recurrence of a disease (e.g., cancer) after an initial response period, e.g., after prior therapy, e.g., cancer therapy (e.g., complete response or partial response). The initial response period may involve the level of malignant cells falling below a certain threshold, for example, below 20%, 15%, 10%, 5%, 4%, 3%, 2%, or 1%. Recurrence may involve the level of malignant cells reaching values above a certain threshold level, for example, above 20%, 1%, 10%, 5%, 4%, 3%, 2%, or 1%. For example, in the context of B-ALL, reoccurrence may involve, for example, reoccurrence of blasts in the blood, bone marrow (>5%), or any extramedullary area after a complete response. In this context, the overall response may involve <5% of BM blasts. In some instances, a response (e.g., complete response or partial response) may involve the absence of a detectable MRD (minimal residual disease). In some instances, the initial response period is at least 1, 2, 3, 4, 5, or 6 days; at least 1, 2, 3 or 4 weeks; at least 1, 2, 3, 4, 6, 8, 10, or 12 months; or at least 1, 2, 3, 4, or 5 years.

### Method of Treatment

Bruton's tyrosine kinase (BTK) is a non-receptor kinase that play an important role in oncogenic signaling. BTK has been identified as a critical factor for proliferation and survival of leukemic cells in hematologic cancers. Inhibitors of BTK have been developed for cancer treatment. One BTK inhibitor, ibrutinib, was used as first-line treatment for patients with B cell malignancies. By disrupting B-cell signaling pathways, BTK treatment has been associated with a dramatic lymph node response, but eradication of disease and relapse in high-risk disease remain challenges. Especially for patients with TP53 mutation, the outcome of BTK treatment still needs to be improved.

ROR1 is an onco-embryonic tyrosine kinase-like receptor that is re-expressed at high levels on many solid and hematologic cancers such as MCL, CLL, and MZL but not on normal adult tissues. Without being limited to any particular theory, ROR1 binds Wnt5a, resulting in increased tumor growth, survival, metastasis, cancer cell stemness and epithelial mesenchymal transition.

The present disclosure provides a method for treating a cancer or tumor with a TP53 mutation in an individual by targeting both ROR1 and BTK.

In certain embodiments, the method halts progression of the cancer. In certain embodiments, the method halts progression of the cancer for at least 12 months. In certain embodiments, the method halts progression of the cancer at least 24 months. In certain embodiments, the method halts progression of the cancer at least 36 months. In certain embodiments, the method halts progression of the cancer at least 48 months.

### Anti-ROR1 Antibodies

The ROR1 antagonist disclosed herein can be an antibody or an antigen binding fragment thereof that specifically binds to a ROR1 polypeptide. The antibody can be an anti-ROR1 antibody. The anti-ROR1 antibody can be a humanized antibody. The anti-ROR1 antibody or the antigen binding fragment thereof comprises complementarity-determining regions (CDRs), allowing it to bind portions of a ROR1 polypeptide or a fragment thereof. Exemplary anti-ROR1 antibodies are disclosed in U.S. Pat. Nos. 9,217,040 and 9,758,591, which are herein incorporated by references in their entirety.

In some instances, the antibody or the antigen binding fragment thereof comprises a heavy chain CDR1 (HCDR1) comprising the amino acid sequence set forth in SEQ ID NO: 1, a heavy chain CDR2 (HCDR2) comprising the amino acid sequence set forth in SEQ ID NO: 2, a heavy chain CDR3 (HCDR3) comprising the amino acid sequence set forth in SEQ ID NO: 3, a light chain CDR1 (LCDR1) comprising the amino acid sequence set forth in SEQ ID NO: 4, a light chain CDR2 (LCDR2) comprising the amino acid sequence set forth in SEQ ID NO: 5, and/or a light chain CDR3 (LCDR3) comprising the amino acid sequence set forth in SEQ ID NO: 6. In some instances, the antibody comprises a heavy chain CDR1 (HCDR1) comprising the amino acid sequence set forth in SEQ ID NO: 1. In some instances, the antibody comprises a heavy chain CDR2 (HCDR2) comprising the amino acid sequence set forth in SEQ ID NO: 2. In some instances, the antibody comprises a heavy chain CDR3 (HCDR3) comprising the amino acid sequence set forth in SEQ ID NO: 3. In some instances, the antibody comprises a light chain CDR1 (LCDR1) comprising the amino acid sequence set forth in SEQ ID NO: 4. In some instances, the antibody comprises a light chain CDR2 (LCDR2) comprising the amino acid sequence set forth in SEQ ID NO: 5. In some instances, the antibody comprises a light chain CDR3 (LCDR3) comprising the amino acid sequence set forth in SEQ ID NO: 6.

In some instances, the antibody or the antigen binding fragment thereof comprises a heavy chain variable region comprising an amino acid sequence at least about 75% identical to that set forth in SEQ ID NO: 7; and/or a light chain variable region comprising an amino acid sequence at least about 75% identical to that set forth in SEQ ID NO: 8. In some instances, the antibody or the antigen binding fragment thereof comprises a heavy chain variable region comprising an amino acid sequence at least about 80% identical to that set forth in SEQ ID NO: 7; and/or a light chain variable region comprising an amino acid sequence at least about 80% identical to that set forth in SEQ ID NO: 8. In some instances, the antibody or the antigen binding fragment thereof comprises a heavy chain variable region comprising an amino acid sequence at least about 85% identical to that set forth in SEQ ID NO: 7; and/or a light chain variable region comprising an amino acid sequence at least about 85% identical to that set forth in SEQ ID NO: 8. In some instances, the antibody or the antigen binding fragment thereof comprises a heavy chain variable region comprising an amino acid sequence at least about 90% identical to that set forth in SEQ ID NO: 7; and/or a light chain variable region comprising an amino acid sequence at least about 90% identical to that set forth in SEQ ID NO: 8. In some instances, the antibody or the antigen binding fragment thereof comprises a heavy chain variable region comprising an amino acid sequence at least about 95% identical to that set forth in SEQ ID NO: 7; and/or a light chain variable region comprising an amino acid sequence at least about 95% identical to that set forth in SEQ ID NO: 8. In some instances, the antibody or the antigen binding fragment thereof comprises a heavy chain variable region comprising an amino acid sequence at least about 96% identical to that set forth in SEQ ID NO: 7; and/or a light chain variable region comprising an amino acid sequence at least about 96% identical to that set forth in SEQ ID NO: 8. In some instances, the antibody or the antigen binding fragment thereof comprises a heavy chain variable region comprising an amino acid sequence at least about 97% identical to that set forth in SEQ ID NO: 7; and/or a light chain variable region comprising an amino acid sequence at least about 97% identical to that set forth in SEQ ID NO: 8. In some instances, the antibody or the antigen binding fragment thereof comprises a heavy chain variable region comprising an amino acid sequence at least about 98% identical to that set forth in SEQ ID NO: 7; and/or a light chain variable region comprising an amino acid sequence at least about 98% identical to that set forth in SEQ ID NO: 8. In some instances, the antibody or the antigen binding fragment thereof comprises a heavy chain variable region comprising an amino acid sequence at least about 99% identical to that set forth in SEQ ID NO: 7; and/or a light chain variable region comprising an amino acid sequence at least about 99% identical to that set forth in SEQ ID NO: 8. In some instances, the antibody or the antigen binding fragment thereof comprises a heavy chain variable region comprising an amino acid sequence at least about 99.5% identical to that set forth in SEQ ID NO: 7; and/or a light chain variable region comprising an amino acid sequence at least about 99.5% identical to that set forth in SEQ ID NO: 8. In some instances, the antibody or the antigen binding fragment thereof comprises a heavy chain variable region comprising an amino acid sequence set forth in SEQ ID NO: 7; and/or a light chain variable region comprising an amino acid sequence set forth in SEQ ID NO: 8. In some instances, the antibody or the antigen binding fragment thereof comprises a heavy chain variable region consisting of an amino acid sequence set forth in SEQ ID NO: 7; and/or a light chain variable region consisting of an amino acid sequence set forth in SEQ ID NO: 8.

In some instances, the antibody or the antigen binding fragment thereof comprises a heavy chain (HC) comprising an amino acid sequence at least about 75% identical to that set forth in SEQ ID NO: 9; and/or a light chain (LC) comprising an amino acid sequence at least about 75% identical to that set forth in SEQ ID NO: 10. In some instances, the antibody or the antigen binding fragment thereof comprises a heavy chain (HC) comprising an amino acid sequence at least about 80% identical to that set forth in SEQ ID NO: 9; and/or a light chain (LC) comprising an amino acid sequence at least about 80% identical to that set forth in SEQ ID NO: 10. In some instances, the antibody or the antigen binding fragment thereof comprises a heavy chain (HC) comprising an amino acid sequence at least about 85% identical to that set forth in SEQ ID NO: 9; and/or a light chain (LC) comprising an amino acid sequence at least about 85% identical to that set forth in SEQ ID NO: 10. In some instances, the antibody or the antigen binding fragment thereof comprises a heavy chain (HC) comprising an amino acid sequence at least about 90% identical to that set forth in SEQ ID NO: 9; and/or a light chain (LC) comprising an amino acid sequence at least about 90% identical to that set forth in SEQ ID NO: 10. In some instances, the antibody or the antigen binding fragment thereof comprises a heavy chain (HC) comprising an amino acid sequence at least about 95% identical to that set forth in SEQ ID NO: 9; and/or a light chain (LC) comprising an amino acid sequence at least about 95% identical to that set forth in SEQ ID NO: 10. In some instances, the antibody or the antigen binding fragment thereof comprises a heavy chain (HC) comprising an amino acid sequence at least about 96% identical to that set forth in SEQ ID NO: 9; and/or a light chain (LC) comprising an amino acid sequence at least about 96% identical to that set forth in SEQ ID NO: 10. In some instances, the antibody or the antigen binding fragment thereof comprises a heavy chain (HC) comprising an amino acid sequence at least about 97% identical to that set forth in SEQ ID NO: 9; and/or a light chain (LC) comprising an amino acid sequence at least about 97% identical to that set forth in SEQ ID NO: 10. In some instances, the antibody or the antigen binding fragment thereof comprises a heavy chain (HC) comprising an amino acid sequence at least about 98% identical to that set forth in SEQ ID NO: 9; and/or a light chain (LC) comprising an amino acid sequence at least about 98% identical to that set forth in SEQ ID NO: 10. In some instances, the antibody or the antigen binding fragment thereof comprises a heavy chain (HC) comprising an amino acid sequence at least about 99% identical to that set forth in SEQ ID NO: 9; and/or a light chain (LC) comprising an amino acid sequence at least about 99% identical to that set forth in SEQ ID NO: 10. In some instances, the antibody or the antigen binding fragment thereof comprises a heavy chain (HC) comprising an amino acid sequence at least about 99.5% identical to that set forth in SEQ ID NO: 9; and/or a light chain (LC) comprising an amino acid sequence at least about 99.5% identical to that set forth in SEQ ID NO: 10. In some instances, the antibody or the antigen binding fragment thereof comprises a heavy chain (HC) comprising an amino acid sequence set forth in SEQ ID NO: 9; and/or a light chain (LC) comprising an amino acid sequence set forth in SEQ ID NO: 10. In some instances, the antibody or the antigen binding fragment thereof comprises a heavy chain (HC) consisting of an amino acid sequence set forth in SEQ ID NO: 9; and/or a light chain (LC) consisting of an amino acid sequence set forth in SEQ ID NO: 10.

In some instances, the antibody or the antigen binding fragment thereof comprises a heavy chain variable region comprising an amino acid sequence at least about 75% identical to that set forth in SEQ ID NO: 7. In some instances, the antibody or the antigen binding fragment thereof comprises a heavy chain variable region comprising an amino acid sequence at least about 80% identical to that set forth in SEQ ID NO: 7. In some instances, the antibody or the antigen binding fragment thereof comprises a heavy chain variable region comprising an amino acid sequence at least about 85% identical to that set forth in SEQ ID NO: 7. In some instances, the antibody or the antigen binding fragment thereof comprises a heavy chain variable region comprising an amino acid sequence at least about 90% identical to that set forth in SEQ ID NO: 7. In some instances, the antibody or the antigen binding fragment thereof comprises a heavy chain variable region comprising an amino acid sequence at least about 95% identical to that set forth in SEQ ID NO: 7. In some instances, the antibody or the antigen binding fragment thereof comprises a heavy chain variable region comprising an amino acid sequence at least about 96% identical to that set forth in SEQ ID NO: 7. In some instances, the antibody or the antigen binding fragment thereof comprises a heavy chain variable region comprising an amino acid sequence at least about 97% identical to that set forth in SEQ ID NO: 7. In some instances, the antibody or the antigen binding fragment thereof comprises a heavy chain variable region comprising an amino acid sequence at least about 98% identical to that set forth in SEQ ID NO: 7. In some instances, the antibody or the antigen binding fragment thereof comprises a heavy chain variable region comprising an amino acid sequence at least about 99% identical to that set forth in SEQ ID NO: 7. In some instances, the antibody or the antigen binding fragment thereof comprises a heavy chain variable region comprising an amino acid sequence at least about 99.5% identical to that set forth in SEQ ID NO: 7. In some instances, the antibody or the antigen binding fragment thereof comprises a heavy chain variable region comprising an amino acid sequence set forth in SEQ ID NO: 7. In some instances, the antibody or the antigen binding fragment thereof comprises a heavy chain variable region consisting of an amino acid sequence set forth in SEQ ID NO: 7.

In some instances, the antibody or the antigen binding fragment thereof comprises a light chain variable region comprising an amino acid sequence at least about 75% identical to that set forth in SEQ ID NO: 8. In some instances, the antibody or the antigen binding fragment thereof comprises a light chain variable region comprising an amino acid sequence at least about 80% identical to that set forth in SEQ ID NO: 8. In some instances, the antibody or the antigen binding fragment thereof comprises a light chain variable region comprising an amino acid sequence at least about 85% identical to that set forth in SEQ ID NO: 8. In some instances, the antibody or the antigen binding fragment thereof comprises a light chain variable region comprising an amino acid sequence at least about 90% identical to that set forth in SEQ ID NO: 8. In some instances, the antibody or the antigen binding fragment thereof comprises a light chain variable region comprising an amino acid sequence at least about 95% identical to that set forth in SEQ ID NO: 8. In some instances, the antibody or the antigen binding fragment thereof comprises a light chain variable region comprising an amino acid sequence at least about 96% identical to that set forth in SEQ ID NO: 8. In some instances, the antibody or the antigen binding fragment thereof comprises a light chain variable region comprising an amino acid sequence at least about 97% identical to that set forth in SEQ ID NO: 8. In some instances, the antibody or the antigen binding fragment thereof comprises a light chain variable region comprising an amino acid sequence at least about 98% identical to that set forth in SEQ ID NO: 8. In some instances, the antibody or the antigen binding fragment thereof comprises a light chain variable region comprising an amino acid sequence at least about 99% identical to that set forth in SEQ ID NO: 8. In some instances, the antibody or the antigen binding fragment thereof comprises a light chain variable region comprising an amino acid sequence at least about 99.5% identical to that set forth in SEQ ID NO: 8. In some instances, the antibody or the antigen binding fragment thereof comprises a light chain variable region comprising an amino acid sequence set forth in SEQ ID NO: 8. In some instances, the antibody or the antigen binding fragment thereof comprises a light chain variable region consisting of an amino acid sequence set forth in SEQ ID NO: 8.

In some instances, the antibody or the antigen binding fragment thereof comprises a heavy chain (HC) comprising an amino acid sequence at least about 75% identical to that set forth in SEQ ID NO: 9. In some instances, the antibody or the antigen binding fragment thereof comprises a heavy chain (HC) comprising an amino acid sequence at least about 80% identical to that set forth in SEQ ID NO: 9. In some instances, the antibody or the antigen binding fragment thereof comprises a heavy chain (HC) comprising an amino acid sequence at least about 85% identical to that set forth in SEQ ID NO: 9. In some instances, the antibody or the antigen binding fragment thereof comprises a heavy chain (HC) comprising an amino acid sequence at least about 90% identical to that set forth in SEQ ID NO: 9. In some instances, the antibody or the antigen binding fragment thereof comprises a heavy chain (HC) comprising an amino acid sequence at least about 95% identical to that set forth in SEQ ID NO: 9. In some instances, the antibody or the antigen binding fragment thereof comprises a heavy chain (HC) comprising an amino acid sequence at least about 96% identical to that set forth in SEQ ID NO: 9. In some instances, the antibody or the antigen binding fragment thereof comprises a heavy chain (HC) comprising an amino acid sequence at least about 97% identical to that set forth in SEQ ID NO: 9. In some instances, the antibody or the antigen binding fragment thereof comprises a heavy chain (HC) comprising an amino acid sequence at least about 98% identical to that set forth in SEQ ID NO: 9. In some instances, the antibody or the antigen binding fragment thereof comprises a heavy chain (HC) comprising an amino acid sequence at least about 99% identical to that set forth in SEQ ID NO: 9. In some instances, the antibody or the antigen binding fragment thereof comprises a heavy chain (HC) comprising an amino acid sequence at least about 99.5% identical to that set forth in SEQ ID NO: 9. In some instances, the antibody or the antigen binding fragment thereof comprises a heavy chain (HC) comprising an amino acid sequence set forth in SEQ ID NO: 9. In some instances, the antibody or the antigen binding fragment thereof comprises a heavy chain (HC) consisting of an amino acid sequence set forth in SEQ ID NO: 9.

In some instances, the antibody or the antigen binding fragment thereof comprises a light chain (LC) comprising an amino acid sequence at least about 75% identical to that set forth in SEQ ID NO: 10. In some instances, the antibody or the antigen binding fragment thereof comprises a light chain (LC) comprising an amino acid sequence at least about 80% identical to that set forth in SEQ ID NO: 10. In some instances, the antibody or the antigen binding fragment thereof comprises a light chain (LC) comprising an amino acid sequence at least about 85% identical to that set forth in SEQ ID NO: 10. In some instances, the antibody or the antigen binding fragment thereof comprises a light chain (LC) comprising an amino acid sequence at least about 90% identical to that set forth in SEQ ID NO: 10. In some instances, the antibody or the antigen binding fragment thereof comprises a light chain (LC) comprising an amino acid sequence at least about 95% identical to that set forth in SEQ ID NO: 10. In some instances, the antibody or the antigen binding fragment thereof comprises a light chain (LC) comprising an amino acid sequence at least about 96% identical to that set forth in SEQ ID NO: 10. In some instances, the antibody or the antigen binding fragment thereof comprises a heavy chain (LC) comprising an amino acid sequence at least about 97% identical to that set forth in SEQ ID NO: 10. In some instances, the antibody or the antigen binding fragment thereof comprises a light chain (LC) comprising an amino acid sequence at least about 98% identical to that set forth in SEQ ID NO: 10. In some instances, the antibody or the antigen binding fragment thereof comprises a light chain (LC) comprising an amino acid sequence at least about 99% identical to that set forth in SEQ ID NO: 10. In some instances, the antibody or the antigen binding fragment thereof comprises a light chain (LC) comprising an amino acid sequence at least about 99.5% identical to that set forth in SEQ ID NO: 10. In some instances, the antibody or the antigen binding fragment thereof comprises a light chain (LC) comprising an amino acid sequence set forth in SEQ ID NO: 10. In some instances, the antibody or the antigen binding fragment thereof comprises a light chain (LC) consisting of an amino acid sequence set forth in SEQ ID NO: 10.

In some instances, the antibody or antigen biding fragment thereof is zilovertamab (formerly called cirmtuzumab or UC-961 or 99961.1) or an ROR1 binding fragment thereof. The development and structure of zilovertamab is disclosed in U.S. Pat. No. 9,758,591, which is incorporated by reference herein in its entirety and for all purposes.

In some instances, the antibody or antigen biding fragment thereof comprises a humanized heavy chain variable region and a humanized light chain variable region, wherein the humanized heavy chain variable region includes the sequences set forth in SEQ ID NO: 15, SEQ ID NO: 16, and SEQ ID NO:17; and wherein the humanized light chain variable region includes the sequences set forth in SEQ ID NO: 18, SEQ ID NO: 19, and SEQ ID NO:20. An antibody including the amino acid sequences (i.e., CDRs) set forth by SEQ ID NOs: 15, 16, 17, 18, 19, 20 may be referred to herein as antibody D10. The development and use of antibody D10 is disclosed in U.S. Pat. No. 9,217,040 which is incorporated by reference herein in its entirety and for all purposes.

In some instances, the antibody or antigen biding fragment thereof binds to amino acids 130-160 of ROR1 or a fragment thereof. In some instances, the antibody or antigen biding fragment thereof binds a peptide including a glutamic acid at a position corresponding to position 138 of ROR1. In some instances, the antibody or antigen biding fragment thereof specifically binds either the 3′ or middle Ig-like region of the extracellular domain of the ROR1 protein. In some instances, the antibody or antigen biding fragment thereof binds the 3′ end of the Ig-like region of the extracellular domain of ROR1 protein from position 1-147.

In some instances, the antibody or antigen biding fragment thereof inhibits metastasis. The antibody or antigen biding fragment thereof can be an antibody fragment. The antibody can be a human antibody. The antibody can be humanized. The antibody can be a chimeric antibody. The antibody can be a single chain antibody. The antibody can be a single chain fragment variable (scFv). The antibody can be a fragment antigen-binding (Fab). The antibody can be a diabody. The antibody can be a nanobody. The antibody can be a minibody. The antibody can be a heavy chain-only antibody (HCAb). The antibody or antigen biding fragment thereof can also be any equivalent of the antibodies or fragments thereof disclosed herein.

In some instances, the antibody has a binding affinity of about 500 pM to about 6 nM. In some instances, the antibody has a binding affinity of about 550 pM to about 6 nM. In some instances, the antibody has a binding affinity of about 600 pM to about 6 nM. In some instances, the antibody has a binding affinity of about 650 pM to about 6 nM. In some instances, the antibody has a binding affinity of about 700 pM to about 6 nM. In some instances, the antibody has a binding affinity of about 750 pM to about 6 nM. In some instances, the antibody has a binding affinity of about 800 pM to about 6 nM. In some instances, the antibody has a binding affinity of about 850 pM to about 6 nM. In some instances, the antibody has a binding affinity of about 900 pM to about 6 nM. In some instances, the antibody has a binding affinity of about 950 pM to about 6 nM. In some instances, the antibody has a binding affinity of about 1 nM to about 6 nM. In some instances, the antibody has a binding affinity of about 1 nM to about 6 nM. In some instances, the antibody has a binding affinity of about 1.5 nM to about 6 nM. In some instances, the antibody has a binding affinity of about 2 nM to about 6 nM. In some instances, the antibody has a binding affinity of about 2.5 nM to about 6 nM. In some instances, the antibody has a binding affinity of about 3 nM to about 6 nM. In some instances, the antibody has a binding affinity of about 3.5 nM to about 6 nM. In some instances, the antibody has a binding affinity of about 4 nM to about 6 nM. In some instances, the antibody has a binding affinity of about 4.5 nM to about 6 nM. In some instances, the antibody has a binding affinity of about 5 nM to about 6 nM. In some instances, the antibody has a binding affinity of about 5.5 nM to about 6 nM.

In some instances, the antibody has a binding affinity of 500 pM to 6 nM. In some instances, the antibody has a binding affinity of 550 pM to 6 nM. In some instances, the antibody has a binding affinity of 600 pM to 6 nM. In some instances, the antibody has a binding affinity of 650 pM to 6 nM. In some instances, the antibody has a binding affinity of 700 pM to 6 nM. In some instances, the antibody has a binding affinity of 750 pM to 6 nM. In some instances, the antibody has a binding affinity of 800 pM to 6 nM. In some instances, the antibody has a binding affinity of 850 pM to 6 nM. In some instances, the antibody has a binding affinity of 900 pM to 6 nM. In some instances, the antibody has a binding affinity of 950 pM to 6 nM. In some instances, the antibody has a binding affinity of 1 nM to 6 nM. In some instances, the antibody has a binding affinity of 1 nM to 6 nM. In some instances, the antibody has a binding affinity of 1.5 nM to 6 nM. In some instances, the antibody has a binding affinity of 2 nM to 6 nM. In some instances, the antibody has a binding affinity of 2.5 nM to 6 nM. In some instances, the antibody has a binding affinity of 3 nM to 6 nM. In some instances, the antibody has a binding affinity of 3.5 nM to 6 nM. In some instances, the antibody has a binding affinity of 4 nM to 6 nM. In some instances, the antibody has a binding affinity of 4.5 nM to 6 nM. In some instances, the antibody has a binding affinity of 5 nM to 6 nM. In some instances, the antibody has a binding affinity of 5.5 nM to 6 nM.

In some instances, the antibody has a binding affinity of about 500 pM to about 5.5 nM. In some instances, the antibody has a binding affinity of about 500 pM to about 5 nM. In some instances, the antibody has a binding affinity of about 500 pM to about 4.5 nM. In some instances, the antibody has a binding affinity of about 500 pM to about 4 nM. In some instances, the antibody has a binding affinity of about 500 pM to about 3.5 nM. In some instances, the antibody has a binding affinity of about 500 pM to about 3 nM. In some instances, the antibody has a binding affinity of about 500 pM to about 3.5 nM. In some instances, the antibody has a binding affinity of about 500 pM to about 3 nM. In some instances, the antibody has a binding affinity of about 500 pM to about 2.5 nM. In some instances, the antibody has a binding affinity of about 500 pM to about 2 nM. In some instances, the antibody has a binding affinity of about 500 pM to about 1.5 nM. In some instances, the antibody has a binding affinity of about 500 pM to about 1 nM. In some instances, the antibody has a binding affinity of about 500 pM to about 950 pM. In some instances, the antibody has a binding affinity of about 500 pM to about 900 pM. In some instances, the antibody has a binding affinity of about 500 pM to about 850 pM. In some instances, the antibody has a binding affinity of about 500 pM to about 800 pM. In some instances, the antibody has a binding affinity of about 500 pM to about 750 pM. In some instances, the antibody has a binding affinity of about 500 pM to about 700 pM. In some instances, the antibody has a binding affinity of about 500 pM to about 650 pM. In some instances, the antibody has a binding affinity of about 500 pM to about 600 pM. In some instances, the antibody has a binding affinity of about 500 pM to about 550 pM.

In some instances, the antibody has a binding affinity of 500 pM to 5.5 nM. In some instances, the antibody has a binding affinity of 500 pM to 5 nM. In some instances, the antibody has a binding affinity of 500 pM to 4.5 nM. In some instances, the antibody has a binding affinity of 500 pM to 4 nM. In some instances, the antibody has a binding affinity of 500 pM to 3.5 nM. In some instances, the antibody has a binding affinity of 500 pM to 3 nM. In some instances, the antibody has a binding affinity of 500 pM to 3.5 nM. In some instances, the antibody has a binding affinity of 500 pM to 3 nM. In some instances, the antibody has a binding affinity of 500 pM to 2.5 nM. In some instances, the antibody has a binding affinity of 500 pM to 2 nM. In some instances, the antibody has a binding affinity of 500 pM to 1.5 nM. In some instances, the antibody has a binding affinity of 500 pM to 1 nM. In some instances, the antibody has a binding affinity of 500 pM to 950 pM. In some instances, the antibody has a binding affinity of 500 pM to 900 pM. In some instances, the antibody has a binding affinity of 500 pM to 850 pM. In some instances, the antibody has a binding affinity of 500 pM to 800 pM. In some instances, the antibody has a binding affinity of 500 pM to 750 pM. In some instances, the antibody has a binding affinity of 500 pM to 700 pM. In some instances, the antibody has a binding affinity of 500 pM to 650 pM. In some instances, the antibody has a binding affinity of 500 pM to 600 pM. In some instances, the antibody has a binding affinity of 500 pM to 550 pM.

In some instances, the antibody has a binding affinity of about 500 pM. In some instances, the antibody has a binding affinity of 500 pM. In some instances, the antibody has a binding affinity of about 550 pM. In some instances, the antibody has a binding affinity of 550 pM. In some instances, the antibody has a binding affinity of about 600 pM. In some instances, the antibody has a binding affinity of 600 pM. In some instances, the antibody has a binding affinity of about 650 pM. In some instances, the antibody has a binding affinity of 650 pM. In some instances, the antibody has a binding affinity of about 700 pM. In some instances, the antibody has a binding affinity of 700 pM. In some instances, the antibody has a binding affinity of about 750 pM. In some instances, the antibody has a binding affinity of 750 pM. In some instances, the antibody has a binding affinity of about 800 pM. In some instances, the antibody has a binding affinity of 800 pM. In some instances, the antibody has a binding affinity of about 850 pM. In some instances, the antibody has a binding affinity of 850 pM. In some instances, the antibody has a binding affinity of about 900 pM. In some instances, the antibody has a binding affinity of 900 pM. In some instances, the antibody has a binding affinity of about 950 pM. In some instances, the antibody has a binding affinity of 950 pM. In some instances, the antibody has a binding affinity of about 1 nM. In some instances, the antibody has a binding affinity of about 1 nM. In some instances, the antibody has a binding affinity of 1 nM. In some instances, the antibody has a binding affinity of about 1.5 nM. In some instances, the antibody has a binding affinity of 1.5 nM. In some instances, the antibody has a binding affinity of about 2 nM. In some instances, the antibody has a binding affinity of 2 nM. In some instances, the antibody has a binding affinity of about 2.5 nM. In some instances, the antibody has a binding affinity of 2.5 nM. In some instances, the antibody has a binding affinity of about 3 nM. In some instances, the antibody has a binding affinity of 3 nM. In some instances, the antibody has a binding affinity of about 3.5 nM. In some instances, the antibody has a binding affinity of 3.5 nM. In some instances, the antibody has a binding affinity of about 4 nM. In some instances, the antibody has a binding affinity of 4 nM. In some instances, the antibody has a binding affinity of about 4.5 nM. In some instances, the antibody has a binding affinity of 4.5 nM. In some instances, the antibody has a binding affinity of about 5 nM. In some instances, the antibody has a binding affinity of 5 nM. In some instances, the antibody has a binding affinity of about 5.5 nM. In some instances, the antibody has a binding affinity of 5.5 nM. In some instances, the antibody has a binding affinity of about 6 nM. In some instances, the antibody has a binding affinity of 6 nM.

In some instances, the antibody binds to an ROR1 protein with a KD of less than about 40 nM (e.g., 35, 30, 25, 20, 15, 10, 9, 8, 7, 6, 5, 4, 3, 2, 1, 0.5, 0.25, 0.1 nM). In some instances, the antibody binds to an ROR1 protein with a KD of less than 40 nM (e.g., 35, 30, 25, 20, 15, 10, 9, 8, 7, 6, 5, 4, 3, 2, 1, 0.5, 0.25, 0.1 nM). In some instances, the antibody binds to an ROR1 protein with a KD of less than about 35 nM. In some instances, the antibody binds to an ROR1 protein with a KD of less than 35 nM. In some instances, the antibody binds to an ROR1 protein with a KD of less than about 30 nM. In some instances, the antibody binds to an ROR1 protein with a KD of less than 30 nM. In some instances, the antibody binds to an ROR1 protein with a KD of less than about 25 nM. In some instances, the antibody binds to an ROR1 protein with a KD of less than 25 nM. In some instances, the antibody binds to an ROR1 protein with a KD of less than about 20 nM. In some instances, the antibody binds to an ROR1 protein with a KD of less than 20 nM. In some instances, the antibody binds to an ROR1 protein with a KD of less than about 15 nM. In some instances, the antibody binds to an ROR1 protein with a KD of less than 15 nM. In some instances, the antibody binds to an ROR1 protein with a KD of less than about 10 nM. In some instances, the antibody binds to an ROR1 protein with a KD of less than 10 nM. In some instances, the antibody binds to an ROR1 protein with a KD of less than about 9 nM. In some instances, the antibody binds to an ROR1 protein with a KD of less than 9 nM. In some instances, the antibody binds to an ROR1 protein with a KD of less than about 8 nM. In some instances, the antibody binds to an ROR1 protein with a KD of less than 8 nM. In some instances, the antibody binds to an ROR1 protein with a KD of less than about 7 nM. In some instances, the antibody binds to an ROR1 protein with a KD of less than 7 nM. In some instances, the antibody binds to an ROR1 protein with a KD of less than about 6 nM. In some instances, the antibody binds to an ROR1 protein with a KD of less than 6 nM. In some instances, the antibody binds to an ROR1 protein with a KD of less than about 5 nM. In some instances, the antibody binds to an ROR1 protein with a KD of less than 5 nM. In some instances, the antibody binds to an ROR1 protein with a KD of less than about 4 nM. In some instances, the antibody binds to an ROR1 protein with a KD of less than 4 nM. In some instances, the antibody binds to an ROR1 protein with a KD of less than about 3 nM. In some instances, the antibody binds to an ROR1 protein with a KD of less than 3 nM. In some instances, the antibody binds to an ROR1 protein with a KD of less than about 2 nM. In some instances, the antibody binds to an ROR1 protein with a KD of less than 2 nM. In some instances, the antibody binds to an ROR1 protein with a KD of less than about 1 nM. In some instances, the antibody binds to an ROR1 protein with a KD of less than 1 nM. In some instances, the antibody binds to an ROR1 protein with a KD of less than about 0.5 nM. In some instances, the antibody binds to an ROR1 protein with a KD of less than 0.5 nM. In some instances, the antibody binds to an ROR1 protein with a KD of less than about 0.25 nM. In some instances, the antibody binds to an ROR1 protein with a KD of less than 0.25 nM. In some instances, the antibody binds to an ROR1 protein with a KD of less than about 0.1 nM. In some instances, the antibody binds to an ROR1 protein with a KD of less than 0.1 nM.

In some instances, the antibody is zilovertamab, also referred to herein as 99961.1 or UC-961. In some instances, the antibody is D10.

The ROR1 antagonist as disclosed herein can be administered to a subject intravenously. The ROR1 antagonist as disclosed herein can be administered to a subject every 1, 2, 4, or 6 weeks. The ROR1 antagonist can be administered to a subject every 2 weeks. The ROR1 antagonist can be administered to a subject every 4 weeks. The ROR1 antagonist can be an antibody disclosed herein. The antibody can be zilovertamab. In some instances, the antibody is administered to a subject intravenously. The antibody can be administered to a subject every 2 weeks. The antibody can be administered to a subject every 4 weeks. In some instances, the antibody is administered to a subject every 2 weeks for at least 2 administrations. In some instances, the antibody is administered to a subject every 2 weeks for at least 3 administrations. In some instances, the antibody is administered to a subject every 2 weeks for at least 4 administrations. In some instances, the antibody is administered to a subject every 2 weeks for at least 5 administrations. In some instances, the antibody is administered to a subject every 2 weeks for at least 6 administrations. In some instances, the antibody is administered to a subject every 2 weeks for 3 administrations and then every 4 weeks thereafter.

In some instances, the ROR1 antagonist is administered at a dose of between about 200 milligrams and about 800 milligrams. In some instances, the ROR1 antagonist is administered at a dose of between about 220 milligrams and about 780 milligrams. In some instances, the ROR1 antagonist is administered at a dose of between about 240 milligrams and about 760 milligrams. In some instances, the ROR1 antagonist is administered at a dose of between about 260 milligrams and about 740 milligrams. In some instances, the ROR1 antagonist is administered at a dose of between about 280 milligrams and about 720 milligrams. In some instances, the ROR1 antagonist is administered at a dose of between about 300 milligrams and about 700 milligrams. In some instances, the ROR1 antagonist is administered at a dose of between about 320 milligrams and about 680 milligrams. In some instances, the ROR1 antagonist is administered at a dose of between about 340 milligrams and about 660 milligrams. In some instances, the ROR1 antagonist is administered at a dose of between about 360 milligrams and about 640 milligrams. In some instances, the ROR1 antagonist is administered at a dose of between about 380 milligrams and about 620 milligrams. In some instances, the ROR1 antagonist is administered at a dose of between about 400 milligrams and about 600 milligrams. In some instances, the ROR1 antagonist is administered at a dose of between about 420 milligrams and about 580 milligrams. In some instances, the ROR1 antagonist is administered at a dose of between about 440 milligrams and about 560 milligrams. In some instances, the ROR1 antagonist is administered at a dose of between about 550 milligrams and about 650 milligrams. In some instances, the ROR1 antagonist is administered at a dose of about 200 milligrams. In some instances, the ROR1 antagonist is administered at a dose of about 300 milligrams. In some instances, the ROR1 antagonist is administered at a dose of about 400 milligrams. In some instances, the ROR1 antagonist is administered at a dose of about 500 milligrams. In some instances, the ROR1 antagonist is administered at a dose of about 600 milligrams. In some instances, the ROR1 antagonist is administered at a dose of about 650 milligrams.

In some instances, the ROR1 antagonist is administered at a dose of about 600 milligrams intravenously every 2 weeks for 3 administrations and then every 4 weeks thereafter. In some instances, the anti-ROR1 antibody is administered at a dose of about 600 milligrams intravenously every 2 weeks for 3 administrations and then every 4 weeks thereafter. In some instances, the anti-ROR1 antibody zilovertamab is administered at a dose of about 600 milligrams intravenously every 2 weeks for 3 administrations and then every 4 weeks thereafter.

### BTK Inhibitor (BTKi)

The BTK inhibitor (BTKi) disclosed herein can be a small molecule. In some instances, the BTKi is ibrutinib, acalabrutinib, evobrutinib, fenebrutinib, orelabrutinib, rilzabrutinib, tolebrutinib, branebrutinib, nemtabrutinib, elsubrutinib, remibrutinib, vecabrutinib, remibrutinib, spebrutinib tirabrutinib, AC0058TA, pirtobrutinib (LOXO-305), zanubrutinib, Spebrutinib, DTRMWXHS-12, M7583, SN-1011, or TG-1701 In some instances, the BTKi is ibrutinib. In some instances, the BTKi is acalabrutinib. In some instances, the BTKi is tirabrutinib (ONO/GS-4059). In some instances, the BTKi is zanubrutinib (BGB-3111). In some instances, the BTKi is CC-292. In some instances, the BTKi is Spebrutinib (AVL-292).

In some instances, the BTKi comprises ibrutinib, acalabrutinib, evobrutinib, fenebrutinib, orelabrutinib, rilzabrutinib, tolebrutinib, branebrutinib, nemtabrutinib, elsubrutinib, remibrutinib, vecabrutinib, remibrutinib, spebrutinib tirabrutinib, AC0058TA, pirtobrutinib (LOXO-305), zanubrutinib, Spebrutinib, DTRMWXHS-12, M7583, SN-1011, or TG-1701. In some instances, the BTKi comprises ibrutinib. In some instances, the BTKi comprises acalabrutinib. In some instances, the BTKi comprises evobrutinib. In some instances, the BTKi comprises fenebrutinib. In some instances, the BTKi comprises orelabrutinib. In some instances, the BTKi comprises rilzabrutinib. In some instances, the BTKi comprises tolebrutinib. In some instances, the BTKi comprises at least two molecules selected from the group consisting of ibrutinib, acalabrutinib, evobrutinib, fenebrutinib, orelabrutinib, rilzabrutinib, tolebrutinib, branebrutinib, nemtabrutinib, elsubrutinib, remibrutinib, vecabrutinib, remibrutinib, spebrutinib tirabrutinib, AC0058TA, pirtobrutinib (LOXO-305), zanubrutinib, Spebrutinib, DTRMWXHS-12, M7583, SN-1011, or TG-1701. In some instances, the BTKi is ibrutinib. In some instances, the BTKi is acalabrutinib. In some instances, the BTKi is evobrutinib. In some instances, the BTKi is fenebrutinib. In some instances, the BTKi is orelabrutinib. In some instances, the BTKi is rilzabrutinib. In some instances, the BTKi is tolebrutinib.

In some instances, the BTKi comprises ibrutinib, acalabrutinib, evobrutinib, fenebrutinib, orelabrutinib, rilzabrutinib, tolebrutinib, branebrutinib, nemtabrutinib, elsubrutinib, remibrutinib, vecabrutinib, remibrutinib, spebrutinib tirabrutinib, AC0058TA, pirtobrutinib (LOXO-305), zanubrutinib, Spebrutinib, DTRMWXHS-12, M7583, SN-1011, or TG-1701. In some instances, the BTKi comprises branebrutinib. In some instances, the BTKi comprises elsubrutinib. In some instances, the BTKi comprises remibrutinib. In some instances, the BTKi comprises vecabrutinib. In some instances, the BTKi comprises nemtabrutinib. In some instances, the BTKi comprises spebrutinib. In some instances, the BTKi comprises tirabrutinib. In some instances, the BTKi comprises AC0058TA. In some instances, the BTKi comprises LOXO-305. In some instances, the BTKi comprises DTRMWXHS-12. In some instances, the BTKi comprises M7583. In some instances, the BTKi comprises SN-1011. In some instances, the BTKi comprises TG-1701. In some instances, the BTKi comprises at least two molecules selected from the group consisting of ibrutinib, acalabrutinib, evobrutinib, fenebrutinib, orelabrutinib, rilzabrutinib, tolebrutinib, branebrutinib, nemtabrutinib, elsubrutinib, remibrutinib, vecabrutinib, remibrutinib, spebrutinib tirabrutinib, AC0058TA, pirtobrutinib (LOXO-305), zanubrutinib, Spebrutinib, DTRMWXHS-12, M7583, SN-1011, or TG-1701. In some instances, the BTKi is branebrutinib. In some instances, the BTKi is elsubrutinib. In some instances, the BTKi is remibrutinib. In some instances, the BTKi is vecabrutinib. In some instances, the BTKi is spebrutinib. In some instances, the BTKi is tirabrutinib. In some instances, the BTKi is AC0058TA. In some instances, the BTKi is LOXO-305. In some instances, the BTKi is DTRMWXHS-12. In some instances, the BTKi is M7583. In some instances, the BTKi is SN-1011. In some instances, the BTKi is TG-1701. In some instances, the BTKi is nemtabrutinib.

The BTKi as disclosed herein can be administered to a subject orally. The BTKi as disclosed herein can be administered to a subject intravenously. The BTKi as disclosed herein can be administered to a subject daily. The BTKi as disclosed herein can be administered to a subject daily for at least 4 weeks. The BTKi as disclosed herein can be administered to a subject daily for at least 8 weeks. The BTKi as disclosed herein can be administered to a subject daily for at least 12 weeks. The BTKi as disclosed herein can be administered to a subject daily for at least 16 weeks. The BTKi as disclosed herein can be administered to a subject daily for at least 18 weeks. The BTKi as disclosed herein can be administered to a subject daily for at least 24 weeks. The BTKi as disclosed herein can be administered to a subject daily for at least 30 weeks.

In some instances, the BTKi is administered at a dose of between about 280 and about 700 milligrams. In some instances, the BTKi is administered at a dose of between about 300 and about 680 milligrams. In some instances, the BTKi is administered at a dose of between about 320 and about 660 milligrams. In some instances, the BTKi is administered at a dose of between about 340 and about 640 milligrams. In some instances, the BTKi is administered at a dose of between about 360 and about 620 milligrams. In some instances, the BTKi is administered at a dose of between about 380 and about 600 milligrams. In some instances, the BTKi is administered at a dose of between about 400 and about 580 milligrams. In some instances, the BTKi is administered at a dose of between about 420 and about 560 milligrams. In some instances, the BTKi is administered at a dose of about 420 milligrams. In some instances, the BTKi is administered at a dose of about 560 milligrams.

In some instances, the BTKi is administered at a dose of about 20 mg to about 160 mg. In some instances, the BTKi is administered at a dose of about 20 mg to about 25 mg, about 20 mg to about 40 mg, about 20 mg to about 45 mg, about 20 mg to about 50 mg, about 20 mg to about 65 mg, about 20 mg to about 80 mg, about 20 mg to about 90 mg, about 20 mg to about 100 mg, about 20 mg to about 125 mg, about 20 mg to about 150 mg, about 20 mg to about 160 mg, about 25 mg to about 40 mg, about 25 mg to about 45 mg, about 25 mg to about 50 mg, about 25 mg to about 65 mg, about 25 mg to about 80 mg, about 25 mg to about 90 mg, about 25 mg to about 100 mg, about 25 mg to about 125 mg, about 25 mg to about 150 mg, about 25 mg to about 160 mg, about 40 mg to about 45 mg, about 40 mg to about 50 mg, about 40 mg to about 65 mg, about 40 mg to about 80 mg, about 40 mg to about 90 mg, about 40 mg to about 100 mg, about 40 mg to about 125 mg, about 40 mg to about 150 mg, about 40 mg to about 160 mg, about 45 mg to about 50 mg, about 45 mg to about 65 mg, about 45 mg to about 80 mg, about 45 mg to about 90 mg, about 45 mg to about 100 mg, about 45 mg to about 125 mg, about 45 mg to about 150 mg, about 45 mg to about 160 mg, about 50 mg to about 65 mg, about 50 mg to about 80 mg, about 50 mg to about 90 mg, about 50 mg to about 100 mg, about 50 mg to about 125 mg, about 50 mg to about 150 mg, about 50 mg to about 160 mg, about 65 mg to about 80 mg, about 65 mg to about 90 mg, about 65 mg to about 100 mg, about 65 mg to about 125 mg, about 65 mg to about 150 mg, about 65 mg to about 160 mg, about 80 mg to about 90 mg, about 80 mg to about 100 mg, about 80 mg to about 125 mg, about 80 mg to about 150 mg, about 80 mg to about 160 mg, about 90 mg to about 100 mg, about 90 mg to about 125 mg, about 90 mg to about 150 mg, about 90 mg to about 160 mg, about 100 mg to about 125 mg, about 100 mg to about 150 mg, about 100 mg to about 160 mg, about 125 mg to about 150 mg, about 125 mg to about 160 mg, or about 150 mg to about 160 mg, including increments therein. In some instances, the BTKi is administered at a dose of about 20 mg, about 25 mg, about 40 mg, about 45 mg, about 50 mg, about 65 mg, about 80 mg, about 90 mg, about 100 mg, about 125 mg, about 150 mg, or about 160 mg. In some instances, the BTKi is administered at a dose of at least about 20 mg, about 25 mg, about 40 mg, about 45 mg, about 50 mg, about 65 mg, about 80 mg, about 90 mg, about 100 mg, about 125 mg, or about 150 mg. In some instances, the BTKi is administered at a dose of at most about 25 mg, about 40 mg, about 45 mg, about 50 mg, about 65 mg, about 80 mg, about 90 mg, about 100 mg, about 125 mg, about 150 mg, or about 160 mg. In some instances, the BTKi is administered at a dose of about 25 mg to about 480 mg. In some instances, the BTKi is administered at a dose of about 25 mg to about 50 mg, about 25 mg to about 75 mg, about 25 mg to about 100 mg, about 25 mg to about 125 mg, about 25 mg to about 150 mg, about 25 mg to about 160 mg, about 25 mg to about 175 mg, about 25 mg to about 200 mg, about 25 mg to about 320 mg, about 25 mg to about 375 mg, about 25 mg to about 480 mg, about 50 mg to about 75 mg, about 50 mg to about 100 mg, about 50 mg to about 125 mg, about 50 mg to about 150 mg, about 50 mg to about 160 mg, about 50 mg to about 175 mg, about 50 mg to about 200 mg, about 50 mg to about 320 mg, about 50 mg to about 375 mg, about 50 mg to about 480 mg, about 75 mg to about 100 mg, about 75 mg to about 125 mg, about 75 mg to about 150 mg, about 75 mg to about 160 mg, about 75 mg to about 175 mg, about 75 mg to about 200 mg, about 75 mg to about 320 mg, about 75 mg to about 375 mg, about 75 mg to about 480 mg, about 100 mg to about 125 mg, about 100 mg to about 150 mg, about 100 mg to about 160 mg, about 100 mg to about 175 mg, about 100 mg to about 200 mg, about 100 mg to about 320 mg, about 100 mg to about 375 mg, about 100 mg to about 480 mg, about 125 mg to about 150 mg, about 125 mg to about 160 mg, about 125 mg to about 175 mg, about 125 mg to about 200 mg, about 125 mg to about 320 mg, about 125 mg to about 375 mg, about 125 mg to about 480 mg, about 150 mg to about 160 mg, about 150 mg to about 175 mg, about 150 mg to about 200 mg, about 150 mg to about 320 mg, about 150 mg to about 375 mg, about 150 mg to about 480 mg, about 160 mg to about 175 mg, about 160 mg to about 200 mg, about 160 mg to about 320 mg, about 160 mg to about 375 mg, about 160 mg to about 480 mg, about 175 mg to about 200 mg, about 175 mg to about 320 mg, about 175 mg to about 375 mg, about 175 mg to about 480 mg, about 200 mg to about 320 mg, about 200 mg to about 375 mg, about 200 mg to about 480 mg, about 320 mg to about 375 mg, about 320 mg to about 480 mg, or about 375 mg to about 480 mg, including increments therein. In some instances, the BTKi is administered at a dose of about 25 mg, about 50 mg, about 75 mg, about 100 mg, about 125 mg, about 150 mg, about 160 mg, about 175 mg, about 200 mg, about 320 mg, about 375 mg, or about 480 mg. In some instances, the BTKi is administered at a dose of at least about 25 mg, about 50 mg, about 75 mg, about 100 mg, about 125 mg, about 150 mg, about 160 mg, about 175 mg, about 200 mg, about 320 mg, or about 375 mg. In some instances, the BTKi is administered at a dose of at most about 50 mg, about 75 mg, about 100 mg, about 125 mg, about 150 mg, about 160 mg, about 175 mg, about 200 mg, about 320 mg, about 375 mg, or about 480 mg. In some instances, the BTKi is administered at a dose of about 280 mg to about 800 mg. In some instances, the BTKi is administered at a dose of about 280 mg to about 300 mg, about 280 mg to about 340 mg, about 280 mg to about 360 mg, about 280 mg to about 400 mg, about 280 mg to about 420 mg, about 280 mg to about 500 mg, about 280 mg to about 560 mg, about 280 mg to about 580 mg, about 280 mg to about 600 mg, about 280 mg to about 700 mg, about 280 mg to about 800 mg, about 300 mg to about 340 mg, about 300 mg to about 360 mg, about 300 mg to about 400 mg, about 300 mg to about 420 mg, about 300 mg to about 500 mg, about 300 mg to about 560 mg, about 300 mg to about 580 mg, about 300 mg to about 600 mg, about 300 mg to about 700 mg, about 300 mg to about 800 mg, about 340 mg to about 360 mg, about 340 mg to about 400 mg, about 340 mg to about 420 mg, about 340 mg to about 500 mg, about 340 mg to about 560 mg, about 340 mg to about 580 mg, about 340 mg to about 600 mg, about 340 mg to about 700 mg, about 340 mg to about 800 mg, about 360 mg to about 400 mg, about 360 mg to about 420 mg, about 360 mg to about 500 mg, about 360 mg to about 560 mg, about 360 mg to about 580 mg, about 360 mg to about 600 mg, about 360 mg to about 700 mg, about 360 mg to about 800 mg, about 400 mg to about 420 mg, about 400 mg to about 500 mg, about 400 mg to about 560 mg, about 400 mg to about 580 mg, about 400 mg to about 600 mg, about 400 mg to about 700 mg, about 400 mg to about 800 mg, about 420 mg to about 500 mg, about 420 mg to about 560 mg, about 420 mg to about 580 mg, about 420 mg to about 600 mg, about 420 mg to about 700 mg, about 420 mg to about 800 mg, about 500 mg to about 560 mg, about 500 mg to about 580 mg, about 500 mg to about 600 mg, about 500 mg to about 700 mg, about 500 mg to about 800 mg, about 560 mg to about 580 mg, about 560 mg to about 600 mg, about 560 mg to about 700 mg, about 560 mg to about 800 mg, about 580 mg to about 600 mg, about 580 mg to about 700 mg, about 580 mg to about 800 mg, about 600 mg to about 700 mg, about 600 mg to about 800 mg, or about 700 mg to about 800 mg, including increments therein. In some instances, the BTKi is administered at a dose of about 280 mg, about 300 mg, about 340 mg, about 360 mg, about 400 mg, about 420 mg, about 500 mg, about 560 mg, about 580 mg, about 600 mg, about 700 mg, or about 800 mg. In some instances, the BTKi is administered at a dose of at least about 280 mg, about 300 mg, about 340 mg, about 360 mg, about 400 mg, about 420 mg, about 500 mg, about 560 mg, about 580 mg, about 600 mg, or about 700 mg. In some instances, the BTKi is administered at a dose of at most about 300 mg, about 340 mg, about 360 mg, about 400 mg, about 420 mg, about 500 mg, about 560 mg, about 580 mg, about 600 mg, about 700 mg, or about 800 mg. In some instances, the BTKi is administered once daily. In some instances, the BTKi is administered twice daily.

In some instances, the BTKi is administered to a subject orally at a dose of between about 280 and about 700 milligrams. In some instances, the BTKi is administered to a subject orally at a dose of between about 280 and about 700 milligrams, wherein the subject has CLL. In some instances, the BTKi is administered to a subject orally at a dose of between about 280 and about 700 milligrams, wherein the subject has MCL. In some instances, the BTKi is administered to a subject orally at a dose of between about 280 and about 700 milligrams, wherein the subject has MZL. In some instances, the BTKi is administered to a subject orally at a dose of between about 300 and about 680 milligrams. In some instances, the BTKi is administered to a subject orally at a dose of between about 300 and about 680 milligrams, wherein the subject has CLL. In some instances, the BTKi is administered to a subject orally at a dose of between about 300 and about 680 milligrams, wherein the subject has MCL. In some instances, the BTKi is administered to a subject orally at a dose of between about 300 and about 680 milligrams, wherein the subject has MZL. In some instances, the BTKi is administered to a subject orally at a dose of between about 320 and about 660 milligrams. In some instances, the BTKi is administered to a subject orally at a dose of between about 320 and about 660 milligrams, wherein the subject has CLL. In some instances, the BTKi is administered to a subject orally at a dose of between about 320 and about 660 milligrams, wherein the subject has MCL. In some instances, the BTKi is administered to a subject orally at a dose of between about 320 and about 660 milligrams, wherein the subject has MZL. In some instances, the BTKi is administered to a subject orally at a dose of between about 340 and about 640 milligrams. In some instances, the BTKi is administered to a subject orally at a dose of between about 340 and about 640 milligrams, wherein the subject has CLL. In some instances, the BTKi is administered to a subject orally at a dose of between about 340 and about 640 milligrams, wherein the subject has MCL. In some instances, the BTKi is administered to a subject orally at a dose of between about 340 and about 640 milligrams, wherein the subject has MZL. In some instances, the BTKi is administered to a subject orally at a dose of between about 360 and about 620 milligrams. In some instances, the BTKi is administered to a subject orally at a dose of between about 360 and about 620 milligrams, wherein the subject has CLL. In some instances, the BTKi is administered to a subject orally at a dose of between about 360 and about 620 milligrams, wherein the subject has MCL. In some instances, the BTKi is administered to a subject orally at a dose of between about 360 and about 620 milligrams, wherein the subject has MZL. In some instances, the BTKi is administered to a subject orally at a dose of between about 380 and about 600 milligrams. In some instances, the BTKi is administered to a subject orally at a dose of between about 380 and about 600 milligrams, wherein the subject has CLL. In some instances, the BTKi is administered to a subject orally at a dose of between about 380 and about 600 milligrams, wherein the subject has MCL. In some instances, the BTKi is administered to a subject orally at a dose of between about 380 and about 600 milligrams, wherein the subject has MZL. In some instances, the BTKi is administered to a subject orally at a dose of between about 400 and about 580 milligrams. In some instances, the BTKi is administered to a subject orally at a dose of between about 400 and about 580 milligrams, wherein the subject has CLL. In some instances, the BTKi is administered to a subject orally at a dose of between about 400 and about 580 milligrams, wherein the subject has MCL. In some instances, the BTKi is administered to a subject orally at a dose of between about 400 and about 580 milligrams, wherein the subject has MZL. In some instances, the BTKi is administered to a subject orally at a dose of between about 420 and about 560 milligrams. In some instances, the BTKi is administered to a subject orally at a dose of between about 420 and about 560 milligrams, wherein the subject has CLL. In some instances, the BTKi is administered to a subject orally at a dose of between about 420 and about 560 milligrams, wherein the subject has MCL. In some instances, the BTKi is administered to a subject orally at a dose of between about 420 and about 560 milligrams, wherein the subject has MZL.

In some instances, the BTKi is administered to a subject orally at a dose of about 420 milligrams. In some instances, the BTKi is administered to a subject orally at a dose of about 420 milligrams, wherein the subject has CLL. In some instances, the BTKi is administered to a subject orally at a dose of about 420 milligrams, wherein the subject has MCL. In some instances, the BTKi is administered to a subject orally at a dose of about 420 milligrams, wherein the subject has MZL. In some instances, the BTKi is administered to a subject orally at a dose of about 560 milligrams. In some instances, the BTKi is administered to a subject orally at a dose of about 560 milligrams, wherein the subject has CLL. In some instances, the BTKi is administered to a subject orally at a dose of about 560 milligrams, wherein the subject has MCL. In some instances, the BTKi is administered to a subject orally at a dose of about 560 milligrams, wherein the subject has MZL.

### TP53 Mutation

TP53 encodes the tumor-suppressor protein p53. Protein p53 play an important role in various cellular activities including regulation of the cell cycle and apoptosis and promotion of DNA repair in response to cellular stress signals such as DNA damage (E. Campo et al., Haematologica, 2018; 103(12)). The cancer or tumor as disclosed herein comprises a mutation in a gene TP53. TP53 mutations are associated with poor outcome in cancers. In some instances, the mutation is in an exon of the TP53 gene. In some instances, the mutation is in an intron of the TP53 gene. In some instances, the mutation is a nonsense mutation in the TP53 gene. In some instances, the mutation is a missense mutation in the TP53 gene. In some instances, the mutation results in an amino acid insertion, deletion, or substitution in a protein encoded by the TP53 gene. In some instances, the mutation is associated with del(17p). In some instances, the mutation is a splice-site mutation. In some instances, the mutation is in at least one exon from exons 4-8 of TP53. For example, the mutation can be in exon 4, exon 5, exon 6, exon 7, or exon 8 of TP53. In some instances, the mutation results in an aberration in the DNA-binding domain of p53. In some instances, the mutation results in an aberration in the oligomerization domain of p53. In some instances, the mutation results in an aberration in the C-terminal domain of p53. In some instances, the mutation comprises a single mutation as described herein or a combination of the single mutations as described herein. For example, the mutation can comprise a missense mutation and a del(17p) mutation. The mutation in TP53 can comprise a missense mutation. The mutation in TP 53 can comprise a conformational mutations which charges the p53 protein conformation. The mutation can comprise an insertion mutation and a del(17p) mutation. The mutation can comprise a gene deletion mutation and a del(17p) mutation. The mutation can comprise a splice-site mutation and a del(17p) mutation. The mutation can comprise an insertion mutation and a missense mutation. The mutation can comprise a gene deletion mutation and a missense mutation. The mutation can comprise a splice-site mutation and a missense mutation. The mutation can comprise an insertion mutation and a gene deletion mutation. The mutation can comprise an insertion mutation and a splice-site mutation. The mutation can comprise a gene deletion mutation and a splice-site mutation. The mutation in TP53 can comprise any mutation that results in a loss of wildtype p53 function in a cell. The mutation in TP53 can comprise a mutation at a position of R175H, R248Q, R273H, R248W, R273C, R282W, G245S, R249S, or Y220C. The TP53 mutation may be a contact mutant and may result in the loss of DNA binding. The TP53 R248Q mutation may be a hotspot mutation that lies within the DNA-binding domain of the Tp53 protein which results in a loss of DNA binding. The p53 R273H mutation may a hotspot mutation that lies within the DNA binding domain of the Tp53 protein which results in a loss of DNA binding. The p53 R248W mutation may be a hotspot mutation that lies within the DNA binding domain of the Tp53 protein which results in a loss of DNA binding. The p53 R175 mutation may be a hotspot mutation that lies within the DNA binding domain of the Tp53 protein which results in a loss of DNA binding. The p53 R175 mutation may be a hotspot mutation that lies within the DNA binding domain of the Tp53 protein which results in a loss of DNA binding. The p53 G245mutation may be a hotspot mutation that lies within the DNA binding domain of the Tp53 protein which results in a loss of DNA binding. The p53 R249mutation may be a hotspot mutation that lies within the DNA binding domain of the Tp53 protein which results in a loss of DNA binding. The p53 R282mutation may be a hotspot mutation that lies within the DNA binding domain of the Tp53 protein which results in a loss of DNA binding. The TP53 mutation may be a conformational mutations which charges the p53 protein conformation. The TP53 mutation may be a conformational mutations which charges the p53 protein conformation. The p53 R175H mutation and p53 R249S mutation may be conformational mutations, which dramatically change the TP53 conformation.

The methods disclosed herein can comprise determining if the tumor or cancer comprises a mutation in the TP53 gene. A test can be performed on the tumor or cancer to determine if the tumor or cancer comprises a mutation in the TP53 gene. The test can be based either on genomic DNA or mRNA as template. The test can also be based on p53 protein. For example, the test can be immunohistochemistry, immunofluorescence, PCR, RT-PCR, real-time PCR, fluorescence in situ hybridization (FISH), sequencing including next generation sequencing, or genomic arrays, or any combination thereof.

### Methods of Treatment

The methods provided herein are useful for the treatment of cancer or tumor, particular cancer or tumor having a mutation in TP53. In some instances, the methods and compositions as described herein provide effective treatment for cancers expressing ROR1. Disclosed herein is a method of treating a cancer or tumor in an individual, comprising administering to the individual a ROR1 antagonist and a BTKi, wherein the tumor or cancer comprises a mutation in a TP53 gene. The mutation in the TP53 gene can be any mutation or a combination of mutations as disclosed herein.

Further disclosed herein is a method of treating cancer or tumor in a subject in need thereof. The method comprises administering to the subject in need thereof a therapeutically effective amount of a BTKi and a ROR1 antagonist. The BTKi can be a small molecule. The BTKi can be any of the BTKi small molecules as disclosed herein. The ROR1 antagonist can be an anti-ROR1 antibody or antigen binding fragment thereof. The antibody or antigen binding fragment thereof can be any antibody or fragment disclosed herein. In some instances, the BTKi is ibrutinib. In some instances, the ROR1 antagonist is zilovertamab.

### Combination Treatment

Treatment of cancer by targeting both ROR1 and BTK was disclosed in U.S. Pat. No. 10,688,181B, which is incorporated by reference herein by its entirety.

In some instances, the BTKi and the ROR1 antagonist are administered in a combined synergistic amount. In some instances, the BTKi and anti-ROR1 antibody are administered in a combined synergistic amount. A “combined synergistic amount” as used herein refers to the sum of a first amount (e.g., an amount of a BTKi) and a second amount (e.g., an amount of a ROR1 antagonist) that results in a synergistic effect (i.e., an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

In some instances, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the BTKi when used separately from the ROR1 antagonist. In some instances, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the ROR1 antagonist when used separately from the BTKi.

The synergistic effect may be a BTK activity decreasing effect and/or a ROR1 activity decreasing effect. In some instances, synergy between the BTKi and the ROR1 antagonist may result in about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% greater decrease (e.g., decrease of BTK activity or decrease of ROR1 activity) than the sum of the decrease of the BTKi or the ROR1 antagonist when used individually and separately. In some instances, synergy between the BTKi and the ROR1 antagonist may result in 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% greater inhibition of the BTK protein and/or the ROR1 protein than the sum of the inhibition of the BTKi or the ROR1 antagonist when used individually and separately.

The synergistic effect may be a cancer-treating effect such as a lymphoma (i.e. a lymphoma-treating synergistic effect), leukemia (i.e. a leukemia-treating synergistic effect), myeloma (i.e. a myeloma-treating synergistic effect), AML (i.e. a AML-treating synergistic effect), B-ALL (i.e. a B-ALL-treating synergistic effect), T-ALL (i.e. a T-ALL-treating synergistic effect), renal cell carcinoma (i.e. a renal cell carcinoma-treating synergistic effect), colon cancer (i.e. a colon cancer-treating synergistic effect), colorectal cancer (i.e. a colorectal cancer-treating synergistic effect), breast cancer (i.e. a breast cancer-treating synergistic effect), epithelial squamous cell cancer (i.e., epithelial squamous cell cancer-treating synergistic effect), melanoma (i.e., melanoma-treating synergistic effect), stomach cancer (i.e. a stomach cancer-treating synergistic effect), brain cancer (i.e. a brain cancer-treating synergistic effect), lung cancer (i.e. a lung cancer-treating synergistic effect), pancreatic cancer (i.e. a pancreatic cancer-treating synergistic effect), cervical cancer (i.e. a cervical cancer-treating synergistic effect), ovarian cancer (i.e. an ovarian cancer-treating synergistic effect), liver cancer (i.e. a liver cancer-treating synergistic effect), bladder cancer (i.e. a bladder cancer-treating synergistic effect), prostate cancer (i.e. a prostate cancer-treating synergistic effect), testicular cancer (i.e. a testicular cancer-treating synergistic effect), thyroid cancer (i.e. a thyroid cancer-treating synergistic effect), head and neck cancer (i.e. a head and neck cancer-treating synergistic effect), uterine cancer (i.e. an uterine cancer-treating synergistic effect), adenocarcinoma (i.e. an adenocarcinoma-treating synergistic effect), adrenal cancer (i.e. an adrenal cancer-treating synergistic effect), chronic lymphocytic leukemia (i.e. a chronic lymphocytic leukemia-treating synergistic effect), small lymphocytic lymphoma (i.e. a small lymphocytic lymphoma-treating synergistic effect), marginal cell B-Cell lymphoma (i.e. a marginal cell B-Cell lymphoma-treating synergistic effect), Burkitt's Lymphoma (i.e. a Burkitt's Lymphoma-treating synergistic effect), and B cell leukemia (i.e. a B cell leukemia-treating synergistic effect) treating effect. The cancer or tumor disclosed herein may comprise any of the TP53 mutations as disclosed herein.

The methods as disclosed herein can achieve a high overall response rate (ORR) in patients with cancer or tumor comprising a TP53 mutation. In some instances, the methods of combination treatment disclosed herein can achieve a higher ORR in patients with cancer or tumor comprising a TP53 mutation as compared to single treatment with BTKi (such as a Bruton's Kinase Inhibitor BTKI (e.g., ibrutinib)) alone. In some instances, the methods of combination treatment disclosed herein can achieve at least about 50%, about 55%, about 60%, about 65%, about 70%, about 75%, about 80%, or about 85% ORR in patients with cancer or tumor comprising a TP53 mutation. In some instances, the methods of combination treatment disclosed herein can achieve at least about 60%, about 65%, about 70%, about 75%, about 80%, or about 85% ORR in patients with cancer or tumor comprising a TP53 mutation. In some instances, the methods of combination treatment disclosed herein can achieve at least about 80% ORR in patients with cancer or tumor comprising a TP53 mutation.

The methods as disclosed herein can achieve a high complete response rate (CR) in patients with cancer or tumor comprising a TP53 mutation. In some instances, the methods of combination treatment disclosed herein can achieve a higher CR in patients with cancer or tumor comprising a TP53 mutation as compared to single treatment with BTKi (such as ibrutinib) alone. In some instances, the methods of combination treatment disclosed herein can achieve at least about 15%, about 20%, about 25%, about 30%, about 35%, about 40%, about 45%, or about 50% CR in patients with cancer or tumor comprising a TP53 mutation. In some instances, the methods of combination treatment disclosed herein can achieve at least about 5%, about 10%, about 15%, or about 20% CR in patients with cancer or tumor comprising a TP53 mutation. In some instances, the methods of combination treatment disclosed herein can achieve at least about 15% CR in patients with cancer or tumor comprising a TP53 mutation.

The methods as disclosed herein can achieve a high partial response rate (PR) in patients with cancer or tumor comprising a TP53 mutation. In some instances, the methods of combination treatment disclosed herein can achieve a higher PR in patients with cancer or tumor comprising a TP53 mutation as compared to single treatment with BTKi (such as ibrutinib) alone. In some instances, the methods of combination treatment disclosed herein can achieve at least about 30%, about 35%, about 40%, about 45%, about 50%, about 55%, about 60%, about 65%, or about 70% PR in patients with cancer or tumor comprising a TP53 mutation. In some instances, the methods of combination treatment disclosed herein can achieve at least about 60%, about 65%, or about 70% PR in patients with cancer or tumor comprising a TP53 mutation. In some instances, the methods of combination treatment disclosed herein can achieve at least about 65% PR in patients with cancer or tumor comprising a TP53 mutation.

The methods as disclosed herein can achieve a low stable disease rate (SD) in patients with cancer or tumor comprising a TP53 mutation. In some instances, the methods of combination treatment disclosed herein can achieve a lower SD in patients with cancer or tumor comprising a TP53 mutation as compared to single treatment with BTKi (such as ibrutinib) alone. In some instances, the methods of combination treatment disclosed herein can achieve an SD no more than about 15%, about 12%, about 10%, about 8% or about 6% in patients with cancer or tumor comprising a TP53 mutation.

The methods as disclosed herein can achieve a low progressive disease rate (PD) in patients with cancer or tumor comprising a TP53 mutation. In some instances, the methods of combination treatment disclosed herein can achieve a lower PD in patients with cancer or tumor comprising a TP53 mutation as compared to single treatment with BTKi (such as ibrutinib) alone. In some instances, the methods of combination treatment disclosed herein can achieve an PD no more than about 25%, about 20%, about 15%, about 10%, about 9%, about 8%, about 7%, about 6%, or about 5% in patients with cancer or tumor comprising a TP53 mutation.

In some instances, the methods as disclosed herein can achieve an ORR of at least about 60%, about 70%, about 75%, about 80%, about 85%, or about 90% for at least 3, 6, 9, 12, 15, or 26 months. In some instances, the methods as disclosed herein can achieve a CR of at least about 20%, about 25%, about 30%, about 35%, about 40%, or about 45% for at least 3, 6, 9, 12, 15, or 26 months. In some instances, the methods as disclosed herein can achieve a PR of at least about 40%, about 45%, about 50%, about 55%, or about 60% for at least about 3, about 6, about 9, about 12, about 15, or about 26 months.

In some instances, the methods as disclosed herein can achieve a prolonged progression free survival (PFS). In some instances, the methods as disclosed herein can achieve a median PFS of at least about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 months. In some instances, the methods as disclosed herein can achieve a median PFS of at least about 10, about 11, about 12, about 13, about 14, about 15, about 16, about 17, about 18, about 19, or about 20 months. In some instances, the methods as disclosed herein can achieve a median PFS from about 10 to about 20 months. In some instances, the methods as disclosed herein can achieve a median PFS from about 12 to about 18 months. In some instances, the methods as disclosed herein can achieve a median PFS from about 17 months.

The cancer or tumor to be treated can be MCL. The cancer or tumor to be treated can be CLL. The cancer or tumor to be treated can be small cell lymphocytic leukemia (SLL). The cancer or tumor to be treated can be marginal zone lymphoma (MZL). The cancer or tumor to be treated can be diffuse large B cell lymphoma (DLBCL). The cancer or tumor can be an MCL that is a high-risk subtype according to the simplified mantle cell lymphoma international prognostic index.

The cancer or tumor to be treated can be relapsed or refractory. The cancer or tumor to be treated can have failed 1, 2, 3, 4 or more previous treatment courses or interventions intended to treat the cancer or tumor.

The cancer or tumor comprising a TP53 mutation can be MCL. The cancer or tumor comprising a TP53 mutation can be CLL. The cancer or tumor comprising a TP53 mutation can be small cell lymphocytic leukemia (SLL). The cancer or tumor comprising a TP53 mutation can be marginal zone lymphoma (MZL). The cancer or tumor comprising a TP53 mutation can be diffuse large B cell lymphoma (DLBCL). The cancer or tumor comprising a TP53 mutation can be an MCL that is a high-risk subtype according to the simplified mantle cell lymphoma international prognostic index.

The cancer or tumor with the P53 mutation can be relapsed or refractory. The cancer or tumor with the P53 mutation can have failed 1, 2, 3, 4 or more previous treatments intended to treat the cancer or tumor.

The methods as disclosed herein can achieve a high overall response rate (ORR) in patients with MCL comprising a TP53 mutation. In some instances, the methods of combination treatment disclosed herein can achieve a higher ORR in patients with MCL comprising a TP53 mutation as compared to single treatment with BTKi (such as ibrutinib) alone. In some instances, the methods of combination treatment disclosed herein can achieve at least about 50%, about 55%, about 60%, about 65%, about 70%, about 75%, about 80%, or about 85% ORR in patients with MCL comprising a TP53 mutation. In some instances, the methods of combination treatment disclosed herein can achieve at least about 60%, about 65%, about 70%, about 75%, about 80%, or about 85% ORR in patients with MCL comprising a TP53 mutation. In some instances, the methods of combination treatment disclosed herein can achieve at least about 80% ORR in patients with MCL comprising a TP53 mutation.

The methods as disclosed herein can achieve a high complete response rate (CR) in patients with MCL comprising a TP53 mutation. In some instances, the methods of combination treatment disclosed herein can achieve a higher CR in patients with MCL comprising a TP53 mutation as compared to single treatment with BTKi (such as ibrutinib) alone. In some instances, the methods of combination treatment disclosed herein can achieve at least about 15%, about 20%, about 25%, about 30%, about 35%, about 40%, about 45%, or about 50% CR in patients with MCL comprising a TP53 mutation. In some instances, the methods of combination treatment disclosed herein can achieve at least about 5%, about 10%, about 15%, or about 20% CR in patients with MCL comprising a TP53 mutation. In some instances, the methods of combination treatment disclosed herein can achieve at least about 15% CR in patients with MCL comprising a TP53 mutation.

The methods as disclosed herein can achieve a high partial response rate (PR) in patients with MCL comprising a TP53 mutation. In some instances, the methods of combination treatment disclosed herein can achieve a higher PR in MCL or tumor comprising a TP53 mutation as compared to single treatment with BTKi (such as ibrutinib) alone. In some instances, the methods of combination treatment disclosed herein can achieve at least about 30%, about 35%, about 40%, about 45%, about 50%, about 55%, about 60%, about 65%, or about 70% PR in patients with MCL comprising a TP53 mutation. In some instances, the methods of combination treatment disclosed herein can achieve at least about 60%, about 65%, or about 70% PR in patients with MCL comprising a TP53 mutation. In some instances, the methods of combination treatment disclosed herein can achieve at least about 65% PR in patients with MCL comprising a TP53 mutation.

The methods as disclosed herein can achieve a low stable disease rate (SD) in patients with MCL comprising a TP53 mutation. In some instances, the methods of combination treatment disclosed herein can achieve a lower SD in patients with MCL comprising a TP53 mutation as compared to single treatment with BTKi (such as ibrutinib) alone. In some instances, the methods of combination treatment disclosed herein can achieve an SD no more than about 15%, about 12%, about 10%, about 8% or about 6% in patients with MCL comprising a TP53 mutation.

The methods as disclosed herein can achieve a low progressive disease rate (PD) in patients with MCL comprising a TP53 mutation. In some instances, the methods of combination treatment disclosed herein can achieve a lower PD in patients with MCL comprising a TP53 mutation as compared to single treatment with BTKi (such as ibrutinib) alone. In some instances, the methods of combination treatment disclosed herein can achieve an PD no more than about 25%, about 20%, about 15%, about 10%, about 9%, about 8%, about 7%, about 6%, or about 5% in patients with MCL comprising a TP53 mutation.

In some instances, the methods as disclosed herein can achieve an ORR of at least about 60%, about 70%, about 75%, about 80%, about 85%, or about 90% for at least about 3, about 6, about 9, about 12, about 15, or about 26 months for patients with MCL comprising a TP53 mutation. In some instances, the methods as disclosed herein can achieve a CR of at least about 20%, about 25%, about 30%, about 35%, about 40%, or about 45% for at least about 3, about 6, about 9, about 12, about 15, or about 26 months for patients with MCL comprising a TP53 mutation. In some instances, the methods as disclosed herein can achieve a PR of at least about 40%, about 45%, about 50%, about 55%, or about 60% for at least about 3, about 6, about 9, about 12, about 15, or about 26 months for patients with MCL comprising a TP53 mutation.

In some instances, the methods as disclosed herein can achieve a prolonged progression free survival (PFS) for patients with MCL comprising a TP53 mutation. In some instances, the methods as disclosed herein can achieve a median PFS of at least about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 months for patients with MCL comprising a TP53 mutation. In some instances, the methods as disclosed herein can achieve a median PFS of at least about 10, about 11, about 12, about 13, about 14, about 15, about 16, about 17, about 18, about 19, or about 20 months for patients with MCL comprising a TP53 mutation. In some instances, the methods as disclosed herein can achieve a median PFS from about 10 to about 20 months for patients with MCL comprising a TP53 mutation. In some instances, the methods as disclosed herein can achieve a median PFS from about 12 to about 18 months for patients with MCL comprising a TP53 mutation. In some instances, the methods as disclosed herein can achieve a median PFS from about 17 months for patients with MCL comprising a TP53 mutation.

The BTKi and the ROR1 antagonist may be administered in combination either simultaneously (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of the BTKi and the ROR1 antagonist.

In some instances, the BTKi and the ROR1 antagonist are administered simultaneously or sequentially. In some instances, the BTKi and the ROR1 antagonist are administered simultaneously. In some instances, the BTKi and the ROR1 antagonist are administered sequentially. During the course of treatment, the BTKi and ROR1 antagonist may at times be administered sequentially and at other times be administered simultaneously.

In some instances, where the BTKi and the ROR1 antagonist are administered sequentially, the ROR1 antagonist is administered at a first time point and the BTKi is administered at a second time point, wherein the first time point precedes the second time point. Alternatively, in some instances, where the BTKi and the ROR1 antagonist are administered sequentially, the BTKi is administered at a first time point and the ROR1 antagonist is administered at a second time point, wherein the first time point precedes the second time point.

The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

In instances where the BTKi and ROR1 antagonist are administered simultaneously, the BTKi and ROR1 antagonist may be administered as a mixture. Thus, in some instances, the BTKi and the ROR1 antagonist are admixed prior to administration.

In some instances, the BTKi is administered at an amount of about 1 mg/kg, 2 mg/kg, 5 mg/kg, 10 mg/kg or 15 mg/kg. In some instances, the BTKi is administered at an amount of about 1 mg/kg. In some instances, the BTKi is administered at an amount of 1 mg/kg. In some instances, the BTKi is administered at an amount of about 2 mg/kg. In some instances, the BTKi is administered at an amount of 2 mg/kg. In some instances, the BTKi is administered at an amount of about 5 mg/kg. In some instances, the BTKi is administered at an amount of 5 mg/kg. In some instances, the BTKi is administered at an amount of about 10 mg/kg. In some instances, the BTKi is administered at an amount of 10 mg/kg. In some instances, the BTKi is administered at an amount of about 15 mg/kg. In some instances, the BTKi is administered at an amount of 15 mg/kg. In some instances, the BTKi is administered at an amount of about 420 mg. In some instances, the BTKi is administered at an amount of 420 mg. In some instances, the BTKi is administered at an amount of about 560 mg. In some instances, the BTKi is administered at an amount of 560 mg.

In some instances, the ROR1 antagonist is administered at an amount of about 1 mg/kg, 2 mg/kg, 3 mg/kg, 5 mg/kg or 10 mg/kg. In some instances, the ROR1 antagonist is administered at an amount of about 1 mg/kg. In some instances, the ROR1 antagonist is administered at an amount of 1 mg/kg. In some instances, the ROR1 antagonist is administered at an amount of about 2 mg/kg. In some instances, the ROR1 antagonist is administered at an amount of 2 mg/kg. In some instances, the ROR1 antagonist is administered at an amount of about 3 mg/kg. In some instances, the ROR1 antagonist is administered at an amount of 3 mg/kg. In some instances, the ROR1 antagonist is administered at an amount of about 5 mg/kg. In some instances, the ROR1 antagonist is administered at an amount of 5 mg/kg. In some instances, the ROR1 antagonist is administered at an amount of about 10 mg/kg. In some instances, the ROR1 antagonist is administered at an amount of 10 mg/kg. In some instances, the ROR1 antagonist is administered at an amount of about 600 mg. In some instances, the ROR1 antagonist is administered at an amount of 600 mg.

In some instances, the BTKi is administered at an amount of about 5 mg/kg and the ROR1 antagonist is administered at about 2 mg/kg. In some instances, the BTKi is administered at an amount of 5 mg/kg and the ROR1 antagonist is administered at 2 mg/kg. In some instances, the BTKi is administered at an amount of about 5 mg/kg and the ROR1 antagonist is administered at about 1 mg/kg. In some instances, the BTKi is administered at an amount of 5 mg/kg and the ROR1 antagonist is administered at 1 mg/kg. In some instances, the BTKi is administered at an amount of about 420 mg and the ROR1 antagonist is administered at an amount of about 600 mg. In some instances, the BTKi is administered at an amount of about 560 mg and the ROR1 antagonist is administered at an amount of about 600 mg.

In some instances, the BTKi is administered daily over the course of at least 14 days (e.g., 15, 16, 17, 18, 19, 20, 21, 22, 23, 24, 25, 26, 27, 28, 29, 30, 31, 32, 33, 34, 35, 40, 45, or 50 days). In some instances, the BTKi is administered daily over the course of at least 15 days. In some instances, the BTKi is administered daily over the course of at least 16 days. In some instances, the BTKi is administered daily over the course of at least 17 days. In some instances, the BTKi is administered daily over the course of at least 18 days. In some instances, the BTKi is administered daily over the course of at least 19 days. In some instances, the BTKi is administered daily over the course of at least 20 days. In some instances, the BTKi is administered daily over the course of at least 21 days. In some instances, the BTKi is administered daily over the course of at least 22 days. In some instances, the BTKi is administered daily over the course of at least 23 days. In some instances, the BTKi is administered daily over the course of at least 24 days. In some instances, the BTKi is administered daily over the course of at least 25 days. In some instances, the BTKi is administered daily over the course of at least 26 days. In some instances, the BTKi is administered daily over the course of at least 27 days. In some instances, the BTKi is administered daily over the course of at least 28 days. In some instances, the BTKi is administered daily over the course of at least 29 days. In some instances, the BTKi is administered daily over the course of at least 30 days. In some instances, the BTKi is administered daily over the course of at least 31 days. In some instances, the BTKi is administered daily over the course of at least 32 days. In some instances, the BTKi is administered daily over the course of at least 33 days. In some instances, the BTKi is administered daily over the course of at least 34 days. In some instances, the BTKi is administered daily over the course of at least 35 days. In some instances, the BTKi is administered daily over the course of at least 40 days. In some instances, the BTKi is administered daily over the course of at least 45 days. In some instances, the BTKi is administered daily over the course of at least 50 days.

In some instances, the BTKi is administered daily over the course of about 28 days. In some instances, the BTKi is administered daily over the course of 28 days.

In some instances, the ROR1 antagonist is administered once over the course of about 28 days. In some instances, the ROR1 antagonist is administered once over the course of 28 days.

In some instances, the BTKi is administered orally. In some instances, the BTKi is administered intravenously. In some instances, the ROR1 antagonist is administered intravenously.

In some instances, the BTKi ibrutinib is administered in combination with the ROR1 antagonist zilovertamab, and ibrutinib is administered intravenously at an amount of 1 mg/kg and zilovertamab is administered intravenously at an amount of 1 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 1 mg/kg and zilovertamab is administered intravenously at an amount of 2 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 1 mg/kg and zilovertamab is administered intravenously at an amount of 3 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 1 mg/kg and zilovertamab is administered intravenously at an amount of 5 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 1 mg/kg and zilovertamab is administered intravenously at an amount of 10 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 2 mg/kg and zilovertamab is administered intravenously at an amount of 1 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 2 mg/kg and zilovertamab is administered intravenously at an amount of 2 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 2 mg/kg and zilovertamab is administered intravenously at an amount of 3 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 2 mg/kg and zilovertamab is administered intravenously at an amount of 5 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 2 mg/kg and zilovertamab is administered intravenously at an amount of 10 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 5 mg/kg and zilovertamab is administered intravenously at an amount of 1 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 5 mg/kg and zilovertamab is administered intravenously at an amount of 2 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 5 mg/kg and zilovertamab is administered intravenously at an amount of 3 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 5 mg/kg and zilovertamab is administered intravenously at an amount of 5 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 5 mg/kg and zilovertamab is administered intravenously at an amount of 10 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 10 mg/kg and zilovertamab is administered intravenously at an amount of 1 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 10 mg/kg and zilovertamab is administered intravenously at an amount of 2 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 10 mg/kg and zilovertamab is administered intravenously at an amount of 3 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 10 mg/kg and zilovertamab is administered intravenously at an amount of 5 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 10 mg/kg and zilovertamab is administered intravenously at an amount of 10 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 15 mg/kg and zilovertamab is administered intravenously at an amount of 1 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 15 mg/kg and zilovertamab is administered intravenously at an amount of 2 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 15 mg/kg and zilovertamab is administered intravenously at an amount of 3 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 15 mg/kg and zilovertamab is administered intravenously at an amount of 5 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 15 mg/kg and zilovertamab is administered intravenously at an amount of 10 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 420 mg and zilovertamab is administered intravenously at an amount of 1 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 420 mg and zilovertamab is administered intravenously at an amount of 2 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 420 mg and zilovertamab is administered intravenously at an amount of 3 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 420 mg and zilovertamab is administered intravenously at an amount of 5 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously at an amount of 420 mg and zilovertamab is administered intravenously at an amount of 10 mg/kg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously or orally at an amount of about 420 mg and zilovertamab is administered intravenously at an amount of about 600 mg. In some instances, ibrutinib is administered daily over the course of 28 days and zilovertamab is administered once over the course of 28 days.

In some instances, the BTKi ibrutinib is administered in combination with the ROR1 antagonist zilovertamab, and ibrutinib is administered intravenously or orally at an amount of about 560 mg and zilovertamab is administered intravenously at an amount of about 600 mg. In some instances, ibrutinib is administered daily over the course of 28 days and zilovertamab is administered once over the course of 28 days.

In some instances, the BTKi ibrutinib is administered to a subject in combination with the ROR1 antagonist zilovertamab, and ibrutinib is administered intravenously or orally at an amount of about 420 mg and zilovertamab is administered intravenously at an amount of about 600 mg, wherein the subject has CLL. In some instances, ibrutinib is administered daily over the course of 28 days and zilovertamab is administered once over the course of 28 days.

In some instances, the BTKi ibrutinib is administered to a subject in combination with the ROR1 antagonist zilovertamab, and ibrutinib is administered intravenously or orally at an amount of about 560 mg and zilovertamab is administered intravenously at an amount of about 600 mg, wherein the subject has MCL. In some instances, ibrutinib is administered daily over the course of 28 days and zilovertamab is administered once over the course of 28 days.

In some instances, the BTKi ibrutinib is administered to a subject in combination with the ROR1 antagonist zilovertamab, and ibrutinib is administered intravenously or orally at an amount of about 560 mg and zilovertamab is administered intravenously at an amount of about 600 mg, wherein the subject has MZL. In some instances, ibrutinib is administered daily over the course of 28 days and zilovertamab is administered once over the course of 28 days.

The ROR1 antagonist and the BTKi can be administered separately. The BTKi ibrutinib and the ROR1 antagonist zilovertamab can be administered separately.

In some instances, the BTKi ibrutinib is administered prior to the administration of the ROR1 antagonist zilovertamab. In some instances, the BTKi ibrutinib is administered daily for at least 2 weeks prior to the administration of the ROR1 antagonist zilovertamab. In some instances, the BTKi ibrutinib is administered daily for at least 4 weeks prior to the administration of the ROR1 antagonist zilovertamab. In some instances, the BTKi ibrutinib is administered daily for at least 6 weeks prior to the administration of the ROR1 antagonist zilovertamab. In some instances, the BTKi ibrutinib is administered daily for at least 8 weeks prior to the administration of the ROR1 antagonist zilovertamab. In some instances, the BTKi ibrutinib is administered daily for at least 10 weeks prior to the administration of the ROR1 antagonist zilovertamab. In some instances, the BTKi ibrutinib is administered daily for at least 12 weeks prior to the administration of the ROR1 antagonist zilovertamab. In some instances, the BTKi ibrutinib is administered daily for at least 14 weeks prior to the administration of the ROR1 antagonist zilovertamab. In some instances, the BTKi ibrutinib is administered daily for at least 16 weeks prior to the administration of the ROR1 antagonist zilovertamab. In some instances, the BTKi ibrutinib is administered daily for at least 18 weeks prior to the administration of the ROR1 antagonist zilovertamab.

In some instances, the ROR1 antagonist zilovertamab is administered prior to the administration of the BTKi ibrutinib. In some instances, the ROR1 antagonist zilovertamab is administered once a week for at least 2 weeks prior to the administration of the BTKi ibrutinib. In some instances, the ROR1 antagonist zilovertamab is administered once a week for at least 4 weeks prior to the administration of the BTKi ibrutinib. In some instances, the ROR1 antagonist zilovertamab is administered once a week for at least 8 weeks prior to the administration of the BTKi ibrutinib. In some instances, the ROR1 antagonist zilovertamab is administered twice a week for at least 4 weeks prior to the administration of the BTKi ibrutinib. In some instances, the ROR1 antagonist zilovertamab is administered twice a week for at least 8 weeks prior to the administration of the BTKi ibrutinib. In some instances, the ROR1 antagonist zilovertamab is administered twice a week for at least 12 weeks prior to the administration of the BTKi ibrutinib. In some instances, the ROR1 antagonist zilovertamab is administered twice a week for at least 16 weeks prior to the administration of the BTKi ibrutinib. In some instances, the ROR1 antagonist zilovertamab is administered once four weeks for at least 4 weeks prior to the administration of the BTKi ibrutinib. In some instances, the ROR1 antagonist zilovertamab is administered once four weeks for at least 8 weeks prior to the administration of the BTKi ibrutinib. In some instances, the ROR1 antagonist zilovertamab is administered once four weeks for at least 12 weeks prior to the administration of the BTKi ibrutinib. In some instances, the ROR1 antagonist zilovertamab is administered once four weeks for at least 16 weeks prior to the administration of the BTKi ibrutinib. In some instances, the ROR1 antagonist zilovertamab is administered once four weeks for at least 20 weeks prior to the administration of the BTKi ibrutinib.

In some instances, ibrutinib is daily administered orally at an amount of about 560 mg and zilovertamab is administered intravenously at an amount of about 600 mg once every 2 weeks. In some instances, ibrutinib is daily administered orally at an amount of about 420 mg and zilovertamab is administered intravenously at an amount of about 600 mg once every 2 weeks. In some instances, ibrutinib is daily administered orally at an amount of about 560 mg and zilovertamab is administered intravenously at an amount of about 600 mg once every 4 weeks. In some instances, ibrutinib is daily administered orally at an amount of about 420 mg and zilovertamab is administered intravenously at an amount of about 600 mg once every 4 weeks.

In some instances, ibrutinib is daily administered to a subject at an amount of about 560 mg and zilovertamab is administered at an amount of about 600 mg once every 2 weeks, wherein the subject has MCL or MZL. In some instances, ibrutinib is daily administered to a subject at an amount of about 420 mg and zilovertamab is administered at an amount of about 600 mg once every 2 weeks, wherein the subject has CLL. In some instances, ibrutinib is daily administered to a subject at an amount of about 560 mg and zilovertamab is administered at an amount of about 600 mg once every 4 weeks, wherein the subject has MCL or MZL. In some instances, ibrutinib is daily administered to a subject at an amount of about 420 mg and zilovertamab is administered at an amount of about 600 mg once every 4 weeks, wherein the subject has CLL.

In some instances, ibrutinib is daily administered to a subject at an amount of about 560 mg for at least 4, 8, or 16 weeks and followed by administering zilovertamab at an amount of about 600 mg once every 2 weeks for 3 administration and once every 4 weeks thereafter, wherein the subject has MCL or MZL. In some instances, ibrutinib is daily administered to a subject at an amount of about 420 mg for at least 4, 8, or 16 weeks and followed by administering zilovertamab at an amount of about 600 mg once every 2 weeks for 3 administration and once every 4 weeks thereafter, wherein the subject has CLL.

In some instances, ibrutinib is daily administered to a subject orally at an amount of about 560 mg and zilovertamab is administered intravenously at an amount of about 600 mg once every 2 weeks, wherein the subject has MCL or MZL. In some instances, ibrutinib is daily administered to a subject orally at an amount of about 420 mg and zilovertamab is administered intravenously at an amount of about 600 mg once every 2 weeks, wherein the subject has CLL. In some instances, ibrutinib is daily administered to a subject orally at an amount of about 560 mg and zilovertamab is administered intravenously at an amount of about 600 mg once every 4 weeks, wherein the subject has MCL or MZL. In some instances, ibrutinib is daily administered to a subject orally at an amount of about 420 mg and zilovertamab is administered intravenously at an amount of about 600 mg once every 4 weeks, wherein the subject has CLL.

In some instances, ibrutinib is daily administered to a subject orally at an amount of about 560 mg for at least 4, 8, or 16 weeks and followed by administering zilovertamab intravenously at an amount of about 600 mg once every 2 weeks for 3 administration and once every 4 weeks thereafter, wherein the subject has MCL or MZL. In some instances, ibrutinib is daily administered to a subject orally at an amount of about 420 mg for at least 4, 8, or 16 weeks and followed by administering zilovertamab intravenously at an amount of about 600 mg once every 2 weeks for 3 administration and once every 4 weeks thereafter, wherein the subject has CLL.

### Patient Population

The methods disclosed herein can be used for treating patients with cancer or tumor having a TP53 mutation as disclosed herein. The cancer or tumor may further express ROR1. In some instances, the cancer is lymphoma, leukemia, myeloma, AML, B-ALL, T-ALL, renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, or adrenal cancer. In some instances, the cancer is chronic lymphocytic leukemia (CLL), small lymphocytic lymphoma, marginal cell B-Cell lymphoma, Burkitt's Lymphoma, or B cell leukemia. In some instances, the tumor or the cancer comprises a blood cancer. In some instances, the tumor or the cancer comprises a leukemia or a lymphoma. In some instances, the tumor or the cancer comprises a B cell leukemia or B cell lymphoma. In some instances, the tumor or the cancer comprises CLL. In some instances, the tumor or the cancer comprises small cell lymphocytic leukemia (SLL). In some instances, the tumor or the cancer comprises MZL. In some instances, the tumor or the cancer comprises diffuse large B cell lymphoma (DLBCL). In some instances, the tumor or the cancer comprises MCL. In some instances, the MCL is a high-risk subtype according to the simplified mantle cell lymphoma international prognostic index (sMIPI, E Hoster, et al, Blood, 111 (2) (2008), pp. 558-565, incorporated by reference herein in its entirety). In some instances, the MCL is a high-risk subtype according to sMIPI and comprises a TP53 mutation.

The cancer or tumor comprises at least one mutation in TP53. In some instances, the mutation is in an exon of the TP53 gene. In some instances, the mutation is in an intron of the TP53 gene. In some instances, the mutation is a nonsense mutation in the TP53 gene. In some instances, the mutation is a missense mutation in the TP53 gene. In some instances, the mutation results in an amino acid insertion, deletion, or substitution in a protein encoded by the TP53 gene. In some instances, the mutation is associated with del(17p). In some instances, the mutation is a splice-site mutation. In some instances, the mutation is in at least one exon from exons 4-8 of TP53. For example, the mutation can be in exon 4, exon 5, exon 6, exon 7, or exon 8 of TP53. In some instances, the mutation results in an aberration in the DNA-binding domain of p53. In some instances, the mutation results in an aberration in the oligomerization domain of p53. In some instances, the mutation results in an aberration in the C-terminal domain of p53. In some instances, the mutation comprises a single mutation as described herein or a combination of the single mutations as described herein. For example, the mutation can comprise a missense mutation and a del(17p) mutation. The mutation can comprise an insertion mutation and a del(17p) mutation. The mutation can comprise a gene deletion mutation and a del(17p) mutation. The mutation can comprise a splice-site mutation and a del(17p) mutation. The mutation can comprise an insertion mutation and a missense mutation. The mutation can comprise a gene deletion mutation and a missense mutation. The mutation can comprise a splice-site mutation and a missense mutation. The mutation can comprise an insertion mutation and a gene deletion mutation. The mutation can comprise an insertion mutation and a splice-site mutation. The mutation can comprise a gene deletion mutation and a splice-site mutation. The mutation in TP53 can comprise any mutation that results in a loss of wildtype p53 function in a cell.

The subject may be screened for ROR1 expression before, at the time of, or after administration of the BTKi and ROR1 antagonist. The subject may be further subject to a TP53 mutation screening before, at the time of, or after administration of the BTKi and ROR1 antagonist. In some instances, the methods disclosed herein comprises determining if the tumor or cancer comprises a mutation in the TP53 gene. A test can be performed on the tumor or the cancer to determine if the tumor or cancer comprises a mutation in the TP53 gene. The test can be performed on a biopsy sample of the tumor or the cancer of the subject. The test can be based either on genomic DNA or mRNA as template. The test can also be based on p53 protein. For example, the test can be immunohistochemistry, immunofluorescence, PCR, RT-PCR, real-time PCR, fluorescence in situ hybridization (FISH), sequencing including next generation sequencing, or genomic arrays.

In some instances, the subject is a mammal. In some instances, the subject is a human. In some instances, the subject having a tumor or cancer with a TP53 mutation. In some instances, the subject having a tumor or cancer with a TP53 mutation and ROR1 expression. In some instances, the subject is a relapsed patient from prior treatment of the cancer or tumor. In some instances, the subject is a patient who developed resistance to prior treatment of the cancer or tumor.

### Pharmaceutical Composition

Further provided herein are pharmaceutical compositions comprising a BTKi, a ROR1 antagonist and a pharmaceutically acceptable excipient for use in treating patients having a cancer or tumor, wherein the tumor or cancer comprises a mutation in a TP53 gene. In some instances, the pharmaceutical composition comprises a BTKi, an anti-ROR1 antibody, and a pharmaceutically acceptable excipient. In some instances, the BTKi and the anti-ROR1 antibody are present in a combined synergistic amount, wherein the combined synergistic amount is effective to treat cancer in a subject in need thereof.

The BTKi and ROR1 antagonist included in the pharmaceutical compositions provided herein may be any one of the BTKi and/or ROR1 antagonists described herein. For example, the BTKi may be ibrutinib and the ROR1 antagonist may be zilovertamab. Likewise, pharmaceutical compositions provided herein may be formulated such that the administered amount of BTKi and ROR1 antagonist is any one of the amounts as described herein. For example, the ibrutinib may be present in an amount such that administration of the composition results in a dosage of about 420 mg or about 560 mg and zilovertamab may be present in an amount that results in a dosage of about 600 mg.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited herein.

Additional embodiments are disclosed in further detail in the following examples, which are provided by way of illustration and are not in any way intended to limit the scope of this disclosure or the claims.

### Example 1—Phase 1/2 Clinical Study

### Study Design

In Phase 1 study (Part 1), patients with relapsed/refractory (R/R) mantle cell lymphoma (MCL) and R/R chronic lymphocytic leukemia/small lymphocytic lymphoma (CLL/SLL) were enrolled (CLL n=18, MCL n=12). 2, 4, 8, and 16 mg/kg and 300 mg and 600 mg doses of zilovertamab (formerly known as cirmtuzumab) were evaluated. Ibrutinib was added to the treatment after 1 month at dose of 420 mg for CLL, 560 mg for MCL. The recommended dosing regime was 600 mg of zilovertamab (i.v.) once every 2 weeks by three times and then once every 4 weeks in combination with ibrutinib at approved doses per indication (i.e., 420 mg for CLL, 560 mg for MCL).

In Phase 2 study, patients were divided into two study groups. In study group 1 (Part 2), patients with R/R CLL, R/R MCL, and marginal zone lymphoma (MZL) were enrolled (CLL n=16, MCL n=21). The patients were treated with the recommended dosing regimen from Phase 1 study, that is, zilovertamab (600 mg) and ibrutinib at approved dose (420 mg CLL, 560 mg MCL and MZL). In study group 2 (Part 3), patients with R/R CLL (n=31) were enrolled and randomly treated with either zilovertamab in combination with ibrutinib or ibrutinib alone.

The demography, disease characteristics and disposition of the patient population is provided in Table 1. As shown in Table 1, the patient population included patients with high-risk disease and who were heavily pre-treated. The most common reason for discontinuation was the completion of 2 years of treatment.

TABLE 1
Demography, Disease Characteristics and Disposition of the Patient Population

| | Parts 1 & 2 | | | | Part 3 | | | |
|---|---|---|---|---|---|---|---|---|
| | zilo + | | zilo + | | zilo + | | | |
| | ibrutinib | | ibrutinib | | ibrutinib | | | |
| | MCL | | CLL | | CLL | | ibrutinib | |
| | N = 33 | | N = 34 | | N = 16 | | N = 10 | |
| Demography and Disease Characteristics | | | | | | | | |
| Median Age, years (min, max) | 65 | (45, 85) | 68 | (37, 86) | 67 | (52, 84) | 66 | (53, 73) |
| Male, n (%) | 27 | (81.8) | 26 | (76.5) | 10 | (55.6) | 3 | (30) |
| ECOG 0-1, n (%) | 30 | (90.9) | 34 | (100.0) | 16 | (88.9) | 10 | (100) |
| Median time from diagnosis to study | 1.96 | (0.04, 9.15) | 1.06 | (0.03, 6) | 7.50 | (0.05, 21.85) | 6.95 | (0.05, 13.29) |
| start, years (min, max) | | | | | | | | |
| Ki-67 ≥30%, n (%) | 17 | (51.5) | NA | | NA | | NA | |
| sMIPI Intermediate/High, n (%) | 15 | (45.5) | NA | | NA | | NA | |
| Bulky disease ≥5 cm, n (%) | 8 | (24.2) | NA | | NA | | NA | |
| RAI staging, ≥2, n (%) | NA | | 24 | (70.6) | 12 | (66.7) | 6 | (60.0) |
| LDH >250 U/L, n (%) | NA | | 15 | (44.1) | 9 | (50.0) | 3 | (50.0) |
| Received prior systemic regimens, n (%) | 33 | (100.0) | 22 | (64.7) | 9 | (50.0) | 4 | (40.0) |
| Median number of prior systemic | 1 | (1, 5) | 2.0 | (1, 10)^{b} | 2.0 | (1, 4)^{b} | 2.0 | (1, 6)^{b} |
| regimens (min, max) | | | | | | | | |
| Prior BTK inhibitor, n (%) | 5 | ( 15.2)^{a} | 0 | | 0 | | 1^{a} | (10.0) |
| Prior Transplant/Cell Therapy, n (%) | 8 | (24.2)^{c} | 1 | (2.9)^{d} | 0 | | 0 | |
| p53 Mutation, n (%) | 17 | (51.5)^{e} | 6 | (17.6)^{f} | 4 | (23.5)^{f} | 1 | (10.0)^{f} |
| Study Population | | | | | | | | |
| Patients Enrolled, n | 33 | | 34 | | 21 | | 10 | |
| Safety Population^{g}, n | 33 | | 34 | | 18 | | 10 | |
| Efficacy Population^{h}, n (%) | 27 | (81.8) | 34 | (100) | 16 | (88.9) | 7 | (70.0) |
| Patient Disposition | | | | | | | | |
| Ongoing, n (%) | 17 | (51.5) | 0 | | 3 | (16.7) | 1 | (10.0) |
| Median Duration of zilovertamab | 11.11 | (0.0, 37.1) | 12.03 | (8.3, 36.1) | 23.09 | (0.0, 24.3) | 0 | |
| exposure, months (min, max) | | | | | | | | |
| Median Duration of Follow-up, months | 19.5 | (19.45, 28.53) | 46.3 | (38.68, 43.54) | 29.0 | (22.68, 30.26) | 30 | (19.18, 33.10) |
| (95% CI) | | | | | | | | |
| Discontinued from Treatment, n (%) | 16 | (48.5) | 34 | (100) | 15 | (83.3) | 9 | (90.0) |
| Reason for Study Discontinuation | | | | | | | | |
| Completed 2 years of treatment, n (%) | 13 | (38.2%) | 15 | (44.1) | 8 | (44.4) | 3 | (30.0) |
| Disease Progression, n (%) | 2 | (5.9)^{j} | 1 | (2.9) | 1 | (5.6) | 1 | (10.0) |
| Adverse Event, n (%) | 5 | (14.7) | 4 | (11.8) | 5 | (27.8) | 3 | (30.0) |
| Death, n (%) | 1 | (3.0) | 1 | (2.9) | 0 | | 0 | |
| Other, n (%) | 6^{k} | (18.2) | 13^{l} | (38.2) | 3^{m} | (16.7) | 2ⁿ | (20.0) |

Zilo—zilovertamab;
NA—not applicable;
sMIPI: Simplified Mantle Cell Lymphoma International Prognostic Index;
LDH: lactate dehydrogenase;
^{a}prior BTK inhibitor = ibrutinib;
^{b}Median number of prior systemic regimens based on number of subjects who received prior treatments for CLL = 22;
^{c}Autologous stem cell transplant (n = 8), Allogeneic stem cell transplant (n = 1), CAR-T (n = 1) patients could have received more than one;
^{d}Autologous stem cell transplant (n = 1);
^{e}percentage of p53 mutation based on number of subject assessed for mutation, for MCL = 16;
^{f}percentage of p53 mutation based on number of subject assessed for mutation, for CLL Part 1&2 = 34, CLL Part 3, zilovertamab + ibrutinib = 17, CLL Part 3, ibrutinib alone = 10, p53 mutation status manually calculated for CLL;
^{g}Safety population is comprised of all enrolled subjects who received at least one dose of zilovertamab (or ibrutinib if Part 3 ibrutinib alone arm);
^{h}Efficacy population is comprised of enrolled subjects who have received at least one dose of zilovertamab and have at least one post-baseline tumor assessment;
ⁱNA- not applicable, zilovertamab treatment duration is not limited to 2 years for MCL patients;
^{j}Includes 6 objective disease progression and 1 clinical progression;
^{k}Other reasons for MCL include: initiation of alternative treatment (1), patient request to withdraw (3), thyroid cancer (1), investigator decision (1);
^{l}Other reasons for CLL Parts 1&2 include: initiation of alternative treatment (3), investigator decision (4), patient request (6);
^{m}Other reasons For CLL part 3 zilovertamab + ibrutinib arm include: investigator decision (1), noncompliance (1), patient request (1);
ⁿOther reasons For CLL part 3 ibrutinib alone arm is investigator decision (1).

### Tumor Reduction Efficacy

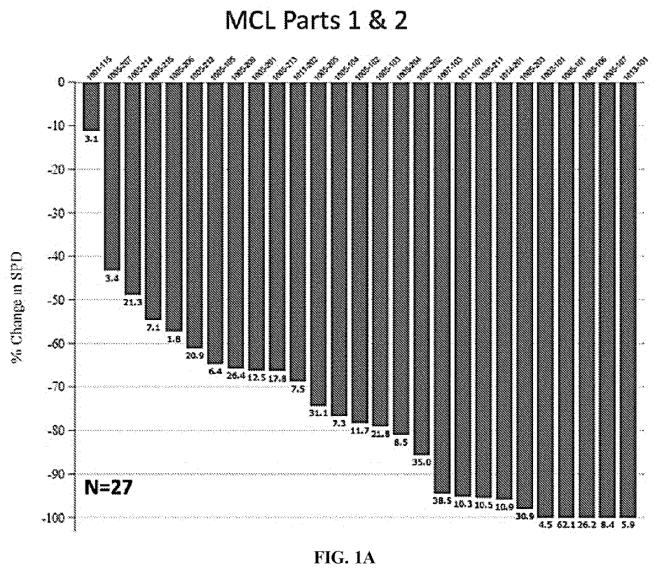

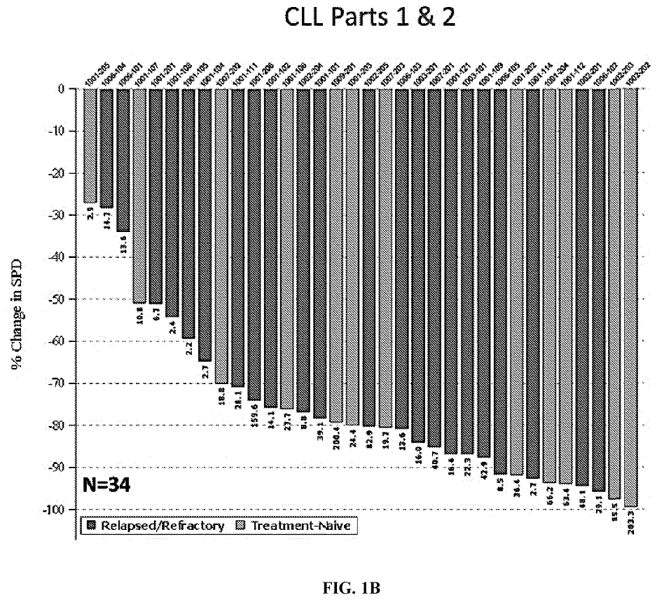

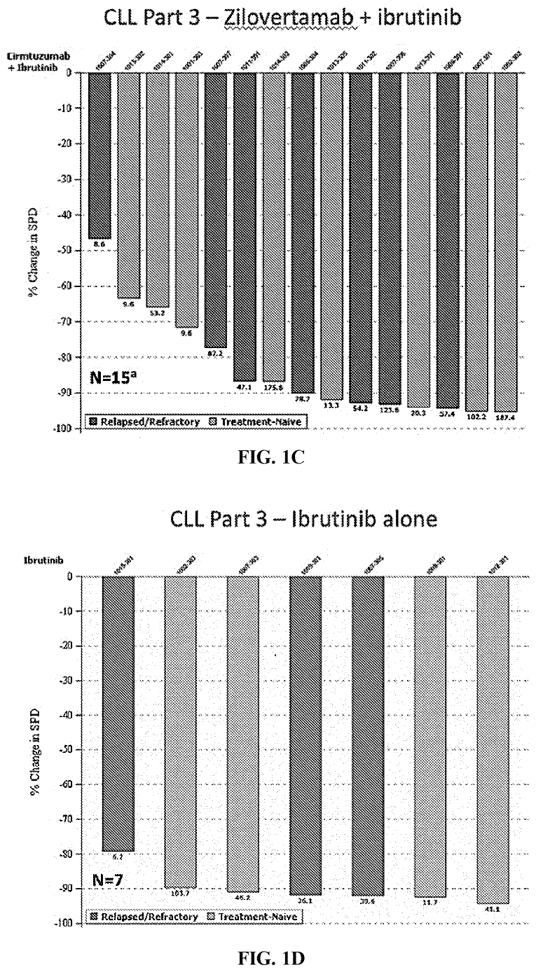

The results show that the combination treatment (zilovertamab+ibrutinib) demonstrated marked reduction in tumor size observed in both MCL & CLL patients (FIG. 1A-1D).

### Safety

The results show that the combination treatment of zilovertamab and ibrutinib is generally well tolerated (Table 2).

TABLE 2
Treatment Emergent Adverse Events in Patients of Parts 1&2

| | | | |
|---|---|---|---|
| | Overall, | Grades | Grades ≥3, |
| N = 33 | n (%) | 1-2, n (%) | n (%) |

MCL Parts 1 & 2

| Fatigue | 18 | (54.5%) | 14 | (42.4%) | 4 | (12.1) |
|---|---|---|---|---|---|---|
| Diarrhea | 19 | (57.6%) | 18 | (54.5%) | 1 | (3.0) |
| Nausea | 11 | (33.3) | 11 | (33.3) | 0 | |
| Contusion | 10 | (30.3) | 10 | (30.3) | 0 | |
| Cough | 10 | (30.3) | 10 | (30.3) | 0 | |
| Dizziness | 9 | (27.3) | 9 | (27.3) | 0 | |
| Stomatitis | 9 | (27.3) | 6 | (18.2) | 3 | (9.1) |
| Anemia | 8 | (24.2) | 5 | (15.2) | 3 | (9.1) |
| Myalgia | 9 | (27.3%) | 8 | (24.2%) | 1 | (3.0) |
| Arthralgia | 8 | (24.2%) | 7 | (21.2%) | 1 | (3.0) |
| Vomiting | 7 | (21.2) | 6 | (18.2) | 1 | (3.0) |

Treatment Emergent Hematological Laboratory Abnormalities

| | | | | | | |
|---|---|---|---|---|---|---|
| Neutrophils decrease | 9 | (27.3%) | 6 | (18.2%) | 3 | (9.1%) |
| Platelets decrease | 23 | (69.7%) | 20 | (60.6%) | 3 | (9.1%) |
| Hemoglobin decrease | 24 | (72.7%) | 21 | (63.6%) | 3 | (9.1%) |

| | | | |
|---|---|---|---|
| | Overall, | Grades | Grades ≥3, |
| CLL N = 34 | n (%) | 1-2, n (%) | n (%) |

CLL Parts 1 & 2

| Contusion | 18 | (52.9%) | 18 | (52.9%) | 0 | |
|---|---|---|---|---|---|---|
| Hypertension | 17 | (50.0%) | 10 | (29.4%) | 7 | (20.6) |
| Diarrhea | 15 | (44.1) | 13 | (38.2) | 2 | (5.9) |
| Upper respiratory | 15 | (44.1) | 15 | (44.1) | 0 | |
| tract infection | | | | | | |
| Fatigue | 14 | (41.2) | 14 | (41.2) | 0 | |
| Muscle Spasms | 11 | (32.4) | 11 | (32.4) | 0 | |
| Onycholclasis | 11 | (32.4) | 11 | (32.4) | 0 | |
| Arthralgia | 10 | (29.4) | 10 | (29.4) | 0 | |
| Cough | 9 | (26.5) | 9 | (26.5) | 0 | |
| Gastroesophageal | 10 | (29.4%) | 10 | (29.4%) | 0 | |
| reflux disease | | | | | | |
| Hypophosphatemia | 9 | (26.5) | 8 | (23.5) | 1 | (2.9) |
| Rash | 10 | (29.4%) | 10 | (29.4%) | 0 | |
| Haematuria | 9 | (26.5%) | 8 | (23.5) | 0 | |
| Dizziness | 7 | (20.6) | 7 | (20.6) | 0 | |
| Dyspnea | 7 | (20.6) | 6 | (17.6) | 1 | (2.9) |
| Headache | 7 | (20.6) | 7 | (20.6) | 0 | |
| Hypercreatinemia | 7 | (20.6) | 6 | (17.6) | 1 | (2.9) |
| Palpitations | 7 | (20.6) | 7 | (20.6) | 0 | |
| Thrombocytopenia | 7 | (20.6) | 6 | (17.6) | 1 | (2.9) |

Treatment Emergent Hematological Laboratory Abnormalities

| | | | | | | |
|---|---|---|---|---|---|---|
| Neutrophils decrease | 16 | (47.1) | 10 | (29.4) | 6 | (17.6) |
| Platelets decrease | 25 | (73.5) | 24 | (70.6) | 1 | (2.9) |

| | | | | | |
|---|---|---|---|---|---|
| Hemoglobin decrease | 25 | (73.5) | 25 | (73.5) | 0 |

The results from Part 3 further shows that the safety profile with the combination treatment (zilovertamab+ibrutinib) was consistent with that with the ibrutinib treatment alone

TABLE 3
Treatment Emergent Adverse Events in Patients of Part 3

| | | | |
|---|---|---|---|
| | Overall, | Grades | Grades ≥3, |
| N = 18 | n (%) | 1-2, n (%) | n (%) |

CCL Part 3: Zilovertamab + ibrutinib

| | | | |
|---|---|---|---|
| Contusion | 7 (38.9) | 7 (38.9) | 0 |
| Back pain | 6 (33.3) | 4 (22.2) | 2 (11.1) |
| Fatigue | 6 (33.3) | 5 (27.8) | 1 (5.6) |
| Cough | 6 (33.3%) | 6 (33.3%) | 0 |
| Diarrhea | 5 (27.8) | 5 (27.8) | 0 |
| Pain in extremity | 5 (27.8) | 5 (27.8) | 0 |
| Thrombocytopenia | 5 (27.8) | 5 (27.8) | 0 |
| Decreased appetite | 4 (22.2) | 4 (22.2) | 0 |
| Dizziness | 4 (22.2) | 4 (22.2) | 0 |
| Dry skin | 4 (22.2) | 4 (22.2) | 0 |
| Dyspnea | 4 (22.2) | 4 (22.2) | 0 |
| Gastroesophageal | 4 (22.2) | 4 (22.2) | 0 |
| reflux disease | | | |
| Hypertension | 4 (22.2) | 4 (22.2) | 1 (5.6) |
| Edema peripheral | 4 (22.2) | 4 (22.2) | 0 |
| Pneumonia | 4 (22.2) | 2 (11.1) | 2 (11.1) |
| Sinusitis | 4 (22.2) | 3 (16.7) | 1 (5.6) |

Treatment Emergent Hematological Laboratory Abnormalities

| | | | |
|---|---|---|---|
| Neutrophils decrease | 6 (33.3) | 5 (27.8) | 1 (5.6) |
| Platelets decrease | 13 (72.2) | 13 (72.2) | 0 |
| Hemoglobin decrease | 16 (88.9) | 16 (88.9) | 0 |

| | | | |
|---|---|---|---|
| | Overall, | Grades | Grades ≥3, |
| N = 10 | n (%) | 1-2, n (%) | n (%) |

CCL Part 3: Ibrutinib

| | | | |
|---|---|---|---|
| Diarrhea | 4 (40.0) | 4 (40.0) | 0 |
| Headache | 4 (40.0) | 3 (30.0) | 1 (10.0) |
| Constipation | 3 (30.0) | 3 (30.0) | 0 |
| Contusion | 3 (30.0) | 3 (30.0) | 0 |
| Dizziness | 3 (30.0) | 3 (30.0) | 0 |
| Hematuria | 3 (30.0) | 3 (30.0) | 0 |
| Nausea | 3 (30.0) | 3 (30.0) | 0 |
| Upper respiratory | 3 (30.0) | 3 (30.0) | 0 |
| tract infection | | | |
| Atrial Fibrillation | 2 (20.0) | 1 (10.0) | 1 (10.0) |
| Back pain | 2 (20.0) | 2 (20.0) | 0 |
| Coronavirus Infection | 2 (20.0) | 1 (10.0) | 1 (10.0) |
| Fall | 2 (20.0) | 2 (20.0) | 0 |
| Fatigue | 2 (20.0) | 2 (20.0) | 0 |
| Hypertension | 2 (20.0) | 1 (10.0) | 1 (10.0) |
| Muscle spasms | 2 (20.0) | 2 (20.0) | 0 |
| Pneumonia | 2 (20.0) | 0 | 2 (20.0) |
| Pruritis | 2 (20.0) | 2 (20.0) | 0 |

Treatment Emergent Hematological Laboratory Abnormalities

| | | | |
|---|---|---|---|
| Neutrophils decrease | 3 (30.0) | 1 (10.0) | 2 (20.0) |
| Platelets decrease | 8 (80.0) | 7 (70.0) | 1 (10.0) |
| Hemoglobin decrease | 7 (70.0) | 6 (60.0) | 1 (10.0) |

### Clinical Response Rates in Subgroups

Clinical response rates in subgroups are provided in Table 4 (for high risk MCL subgroups) and Table 5 (for heavily pre-treated CCL). Data from Table 4 suggests high response rates and durable responses observed in high risk MCL patients. Data from Table 5 suggests promising ORR in heavily pre-treated CLL patients.

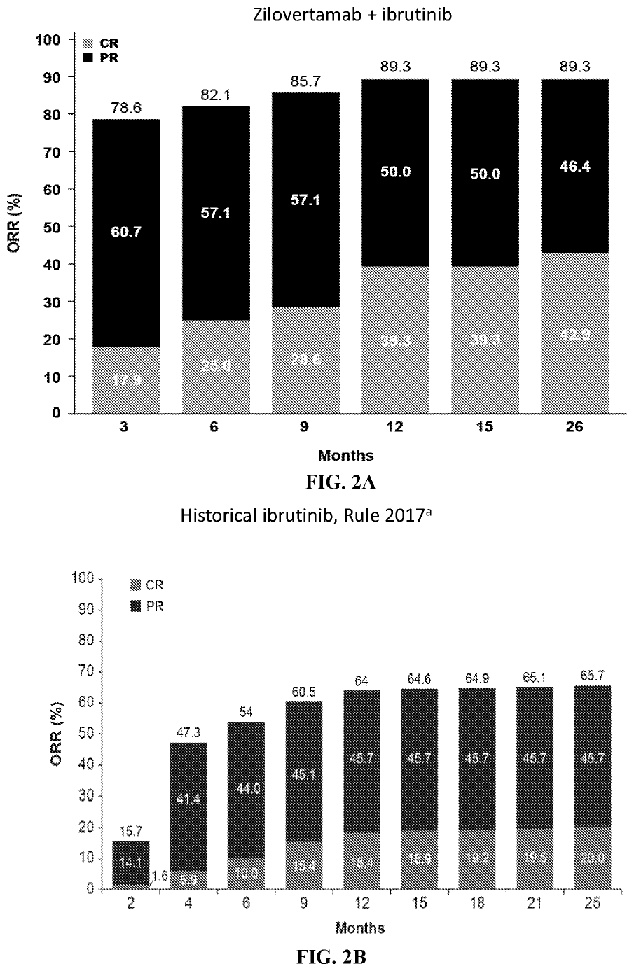

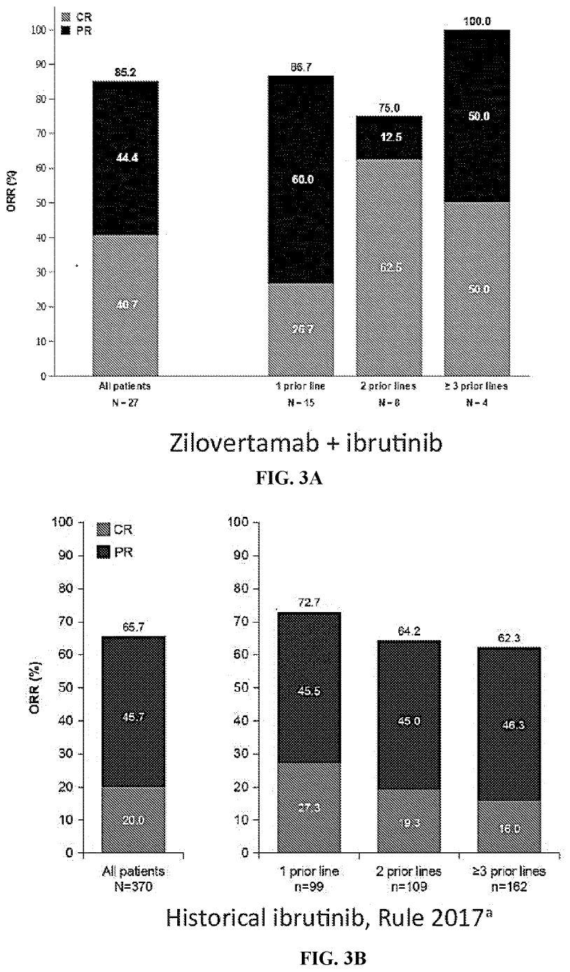

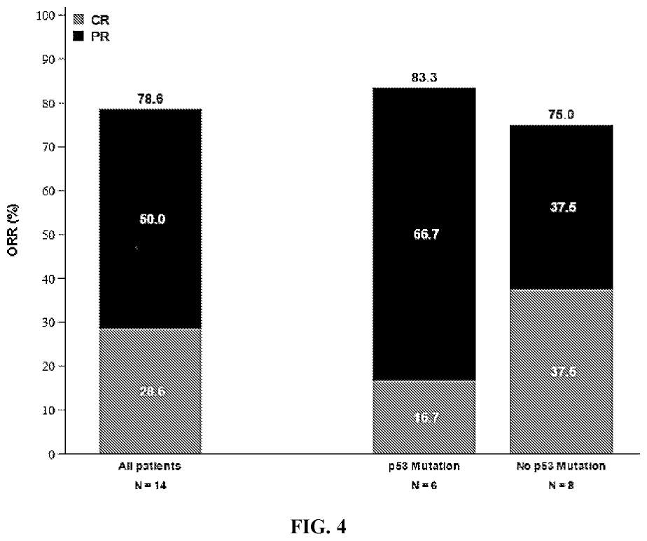

The response rates of the combination treatment were compared with the historical data of ibrutinib treatment alone (Table 6). Data from Table 6 suggests that zilovertamab and ibrutinib combination demonstrated encouraging response rates when compared to historical ibrutinib treatment alone over time (see also FIG. 2A-2B). Further analysis was performed based on prior regimen in MCL subgroup (Table 7 and FIG. 3A-3B) and based on p53 mutation status in the MCL subgroup (Table 8 and FIG. 4).

### Progression-Free Survival in MCL

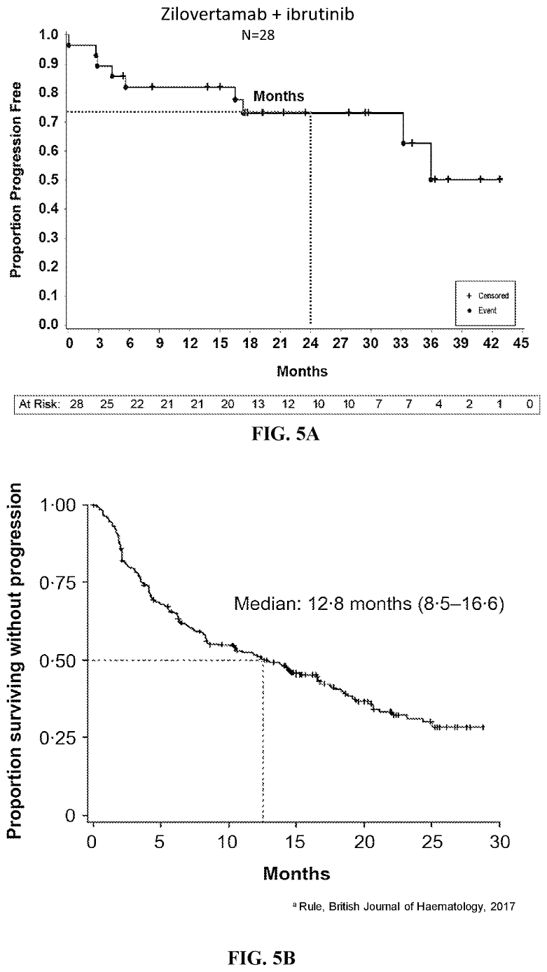

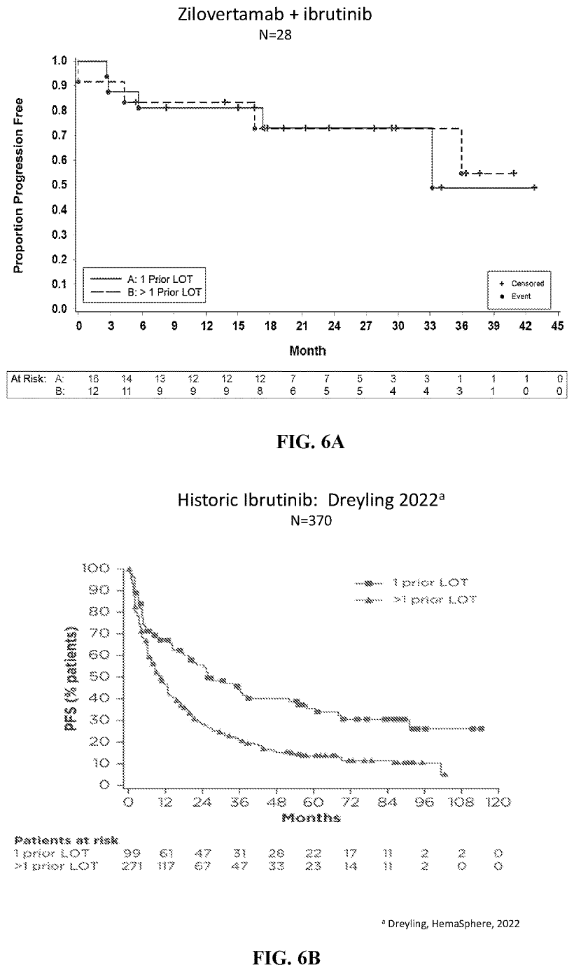

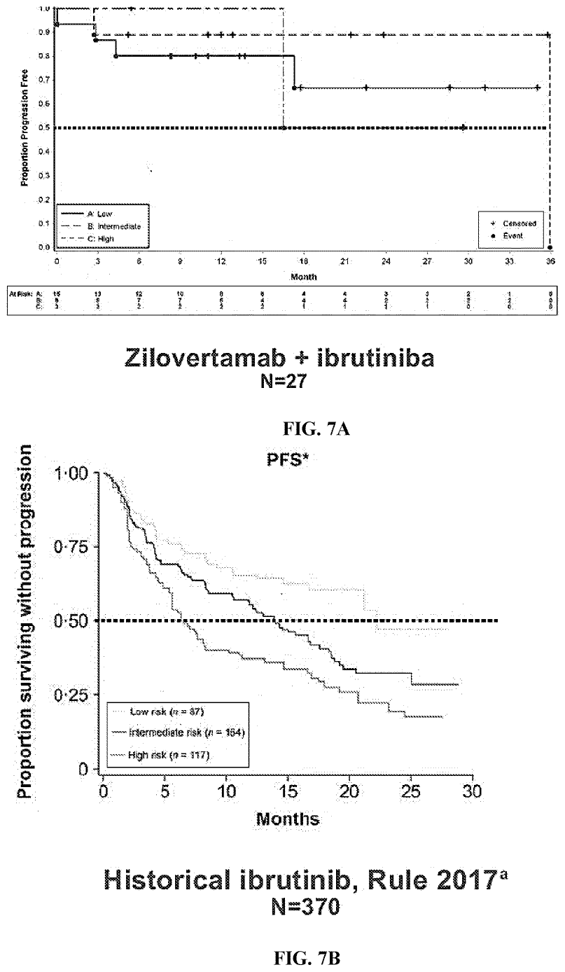

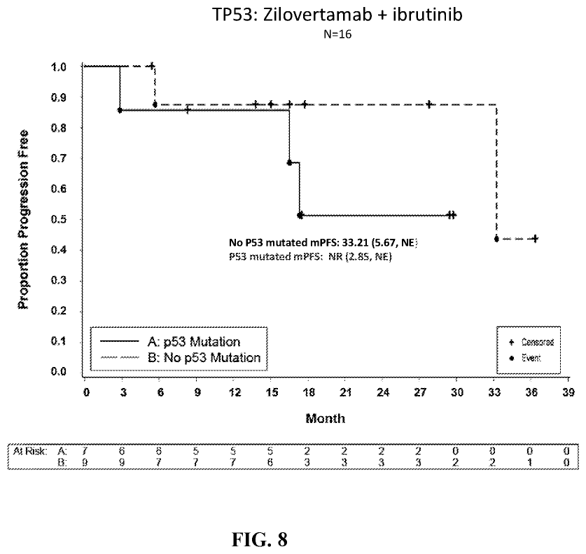

Progression-free Survival (PFS) was compared between the combination treatment (zilovertamab+ibrutinib) and historical ibrutinib treatment alone. The results show that the combination treatment provided favorable PFS benefit compared to historical ibrutinib treatment alone (FIGS. 5A-5B). Further analysis was performed based on prior systemic treatment and the results show that favorable PFS was sustained in heavily pre-treated MCL subjects (FIGS. 6A-6B). Additionally, PFS was assessed based on MIPI subgroups and the results show that favorable PFS was sustained in higher risk MIPI subgroups (FIGS. 7A-7B). Furthermore, PFS was assessed, based on p53 mutation status, in MCL patients (Parts 1&2) treated with the combination treatment (zilovertamab+ibrutinib) as compared to historical ibrutinib treatment alone (Table 9 and FIG. 8). The results suggest that even with p53 mutation, increase in median PFS was observed when compared with the historical ibrutinib treatment.

### Progression-Free Survival in CLL

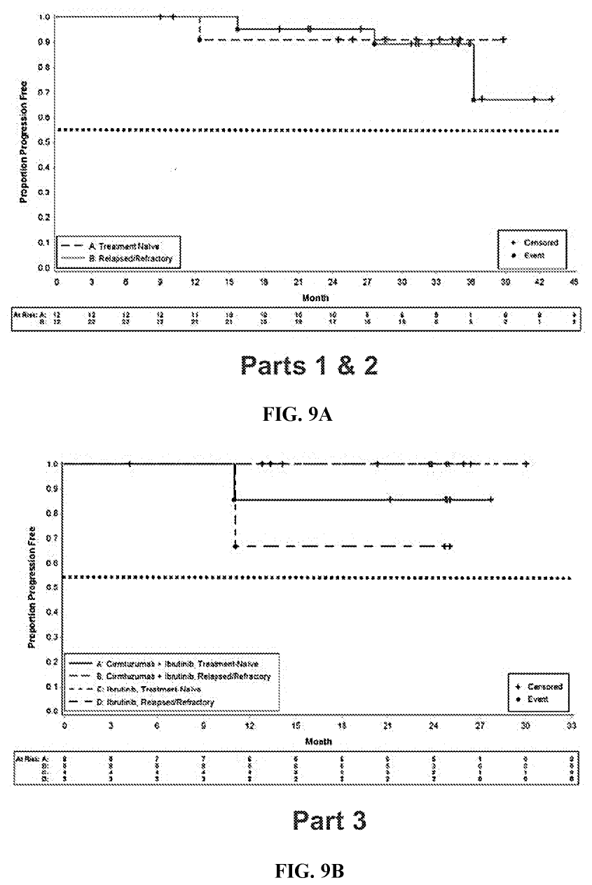

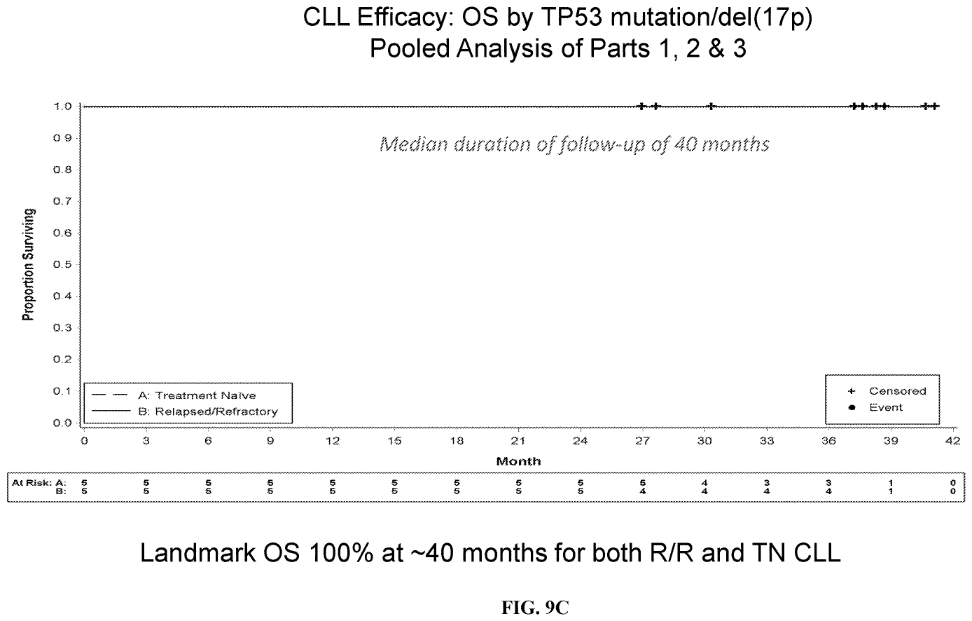

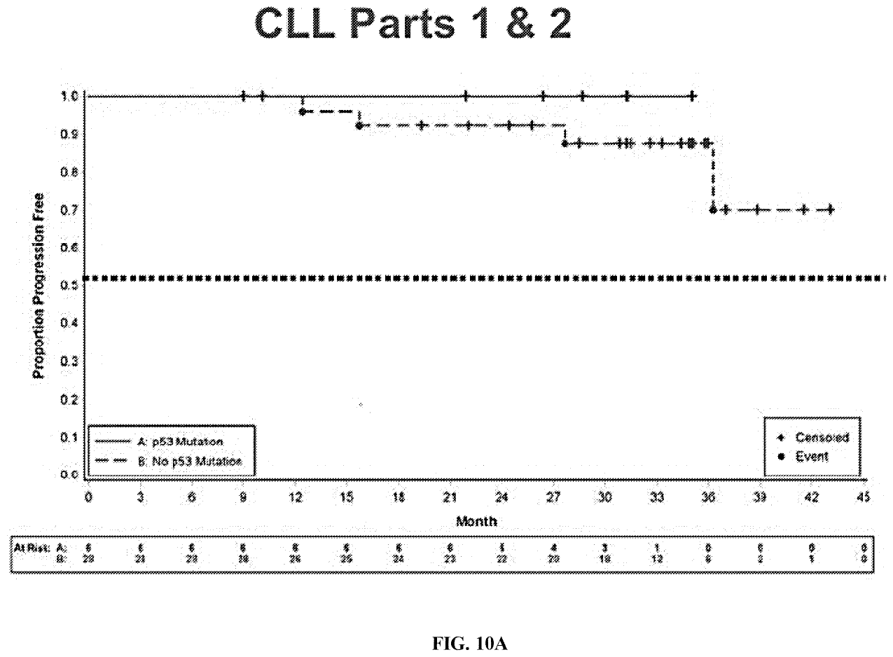

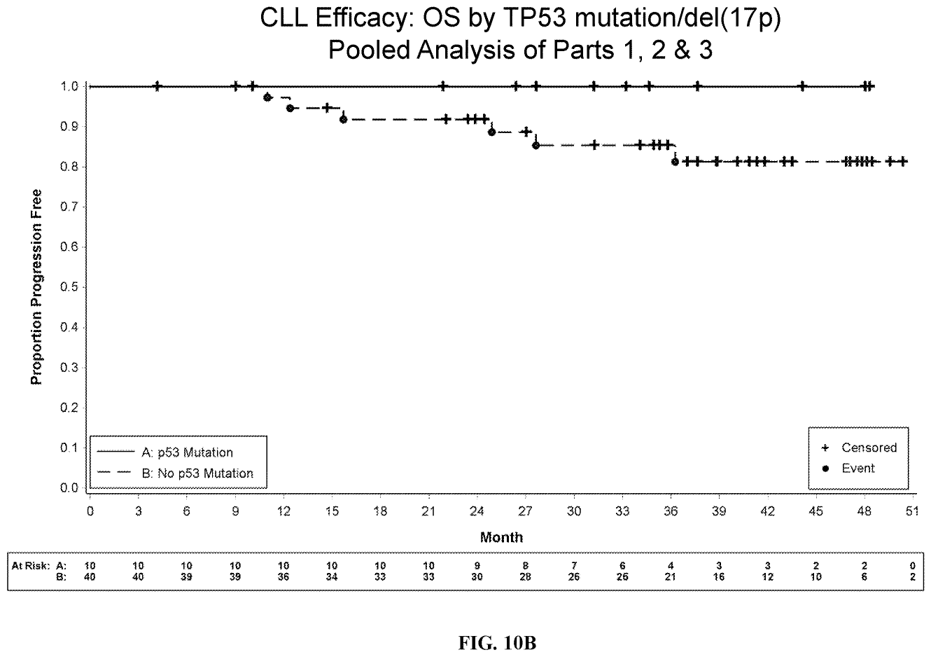

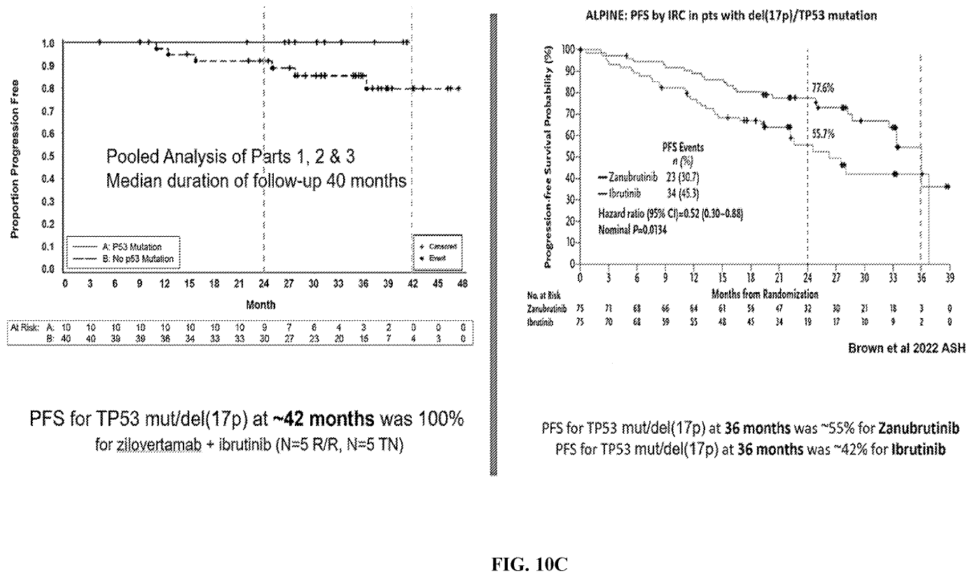

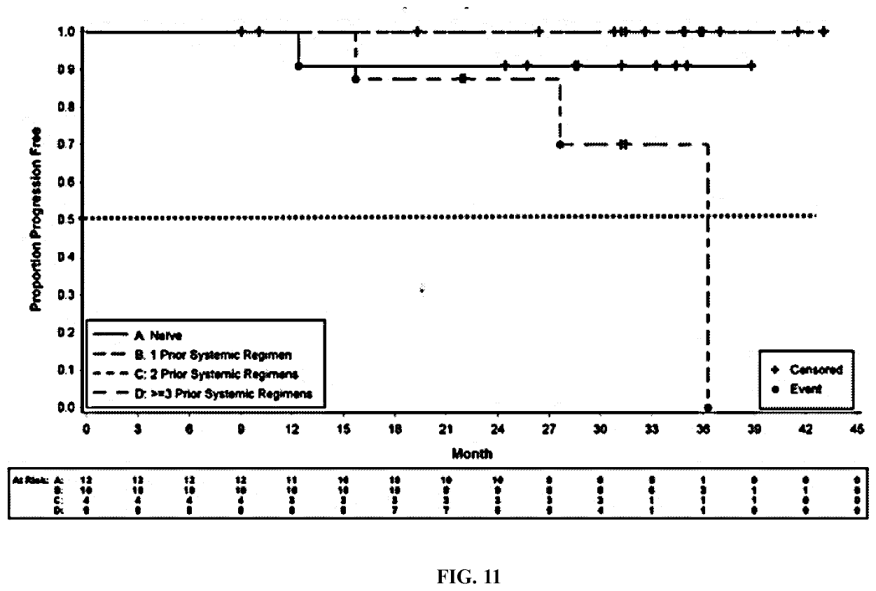

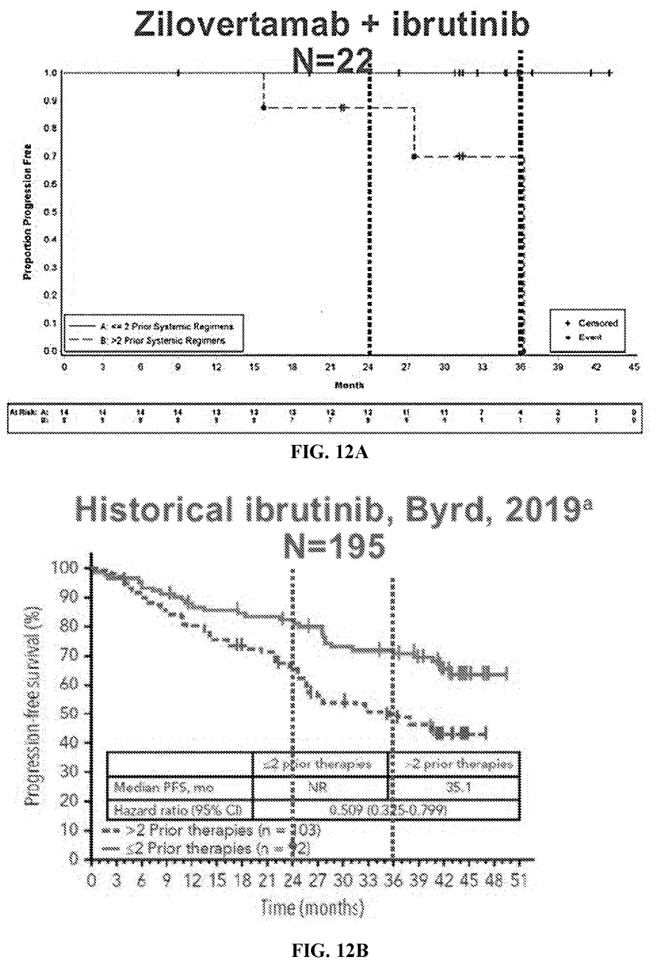

PFS was measured in CLL patients and the results suggest that PFS was not reached at 23+months in both treatment naïve and relapsed/refractory CLL patients (Table 10 and FIGS. 9A-9C). As is shown in FIG. 9C, a 100% PFS rate was observed in both TN and R/R subjects with p53 mutation. Next, PFS was assessed based on p53 mutation status (FIGS. 10A-10C). As is shown in FIG. 10C, a 100% PFS rate was observed in subjects with p53 mutation across parts 1, 2, and 3; and outperformed either ibrutinib or zanubrutinib alone. The results suggest that PFS was not reached at 33+months with or without p53 mutation in CLL patients (Parts 1&2) or in pooled analysis with Part 3 patients receiving combination treatment. Furthermore, PFS was assessed in CLL patients by prior systemic treatment and results suggest that favorable PFS was maintained in heavily pre-treated CLL subjects (Table 11 and FIG. 11). Next, Landmark PFS was assessed by prior systemic treatment and results show that combination treatment (zilovertamab+ibrutinib) demonstrated encouraging Landmark PFS based on number of prior lines of therapy compared to historical ibrutinib treatment alone (FIGS. 12A-12B and Table 12).

Finally, overall survival (OS) was measured in both MCL and CLL patients on combination treatment compared to on ibrutinib alone. The results show that median OS was not reached for MCL and CLL patients on combination treatment or on ibrutinib alone (Table 13). Further, high response rates and durable responses were observed in CLL patients with TP53 mutation/del 17p (Table 14).

Based on the results, combination treatment with zilovertamab+a Bruton's Kinase Inhibitor BTKI (e.g., ibrutinib) is generally well tolerated, with a safety profile similar to that of a Bruton's Kinase Inhibitor BTKI (e.g., ibrutinib) alone. In MCL patients, Grade 3 or 4 neutrophil decrease was seen in 9.1% of patients on the combination treatment, compared to 29% for patients treated with a Bruton's Kinase Inhibitor BTKI (e.g., ibrutinib) alone. The ORR was 89.3%, CRR 43% and median DOR 34.1 months for patients with R/R MCL on zilovertamab+ibrutinib. The PFS rate for Zilovertamab+Ibrutinib was ~95% at 24 months in patients with R/R CLL (median 2 prior LOT) and the PFS rate was 100% at ~42 months for patients with TP53 mutations/del(17p) on zilovertamab+ibrutinib. The data suggest robust efficacy of combination treatment of zilovertamab+a Bruton's Kinase Inhibitor BTKI (e.g., ibrutinib) across multiple high-risk subpopulations compared to a Bruton's Kinase Inhibitor BTKI (e.g., ibrutinib) treatment alone in patients with MCL or CLL. Prolonged PFS was observed for treated patients with MCL or CLL who had TP53 mutations and/or were in high-risk subgroups. High objective response rates and durable responses were observed in heavily pre-treated patients with MCL who were treated with combination treatment of zilovertamab+a Bruton's Kinase Inhibitor BTKI (e.g., ibrutinib).

TABLE 4
Clinical Response Rates in High Risk MCL Subgroups

| | | | | Prior | Bulky | | | | | | | |
|---|---|---|---|---|---|---|---|---|---|---|---|---|
| | | Ki67 ≥ | Prior | SCT +/− | disease | Low | Int | High | 1 Prior | 2 Prior | ≥3 Prior | p53 |
| | Overall | 30% | BTKi^{a} | CAR-T^{b} | (≥5 cm) | sMIPI | sMIPI | sMIPI | Regimen | Regimens | Regimens | mutation |
| | N = 28 | N = 14 | N = 5 | N = 7 | N = 5 | N = 16 | N = 9 | N = 3 | N = 15 | N = 8 | N = 4 | N = 7 |
| ORR, n | 6 | 12 | 5 | 7 | 5 | 15 | 8 | 2 | 14 | 7 | 4 | 6 |
| (%) | | (85.7) | (100.0%) | (100.0) | (100.0) | (93.8%) | (88.9) | (66.7) | (87.5%) | (87.5%) | (100.0) | (85.7) |
| CR, n | 12 | 5^{c} | 3 | 5 | 3 | 7 | 5^{c} | 1 | 4^{c} | 6 | 2 | 1 |
| (%) | (42.9%) | (35.7) | (60.0%) | (71.4) | (60.0%) | (43.8%) | (55.6) | (33.3) | (2.5) | (75.0%) | (50.0) | (14.3) |
| PR, n | 13 | 7 | 2 | 2 | 2 | 8 | 3 | 1 | 10 | 1 | 2 | 5 |
| (%) | (46.4%) | (50.0) | (40.0%) | (28.6) | (40.0%) | (50.0%) | (33.3) | (33.3) | (62.5%) | (12.5%) | (50.0) | (71.4) |
| SD, n | 1 | | 0 | 0 | 0 | 0 | 0 | 1 | 0 | 1 | 0 | 0 |
| (%) | (3.6%) | | | | | | | (33.3) | | (12.5%) | | |
| PD, n | 2 | 2 | 0 | 0 | 0 | 1 | 1 | 0 | 2 | 0 | 0 | 1 |
| (%) | (7.1%) | (14.3) | | | | (6.3%) | (11.1) | | (12.5%) | | | (14.3) |
| Median | 34.13 | NR | 13.67 | 34.13 | 23.90 | NR | 34.13 | NR | 30.46 | NR | 34.13 | 13.84 |
| Duration | (13.84, | (13.67, | (11.93, | (13.84, | (11.93, | (11.93, | (NE) | (13.84, | (11.93, | (1.51, | (13.84, | (11.93, |
| of | NE) | NE) | NE) | NE) | NE) | NE) | | NE) | NE) | NE) | NE) | NE) |
| Response, | | | | | | | | | | | | |
| months | | | | | | | | | | | | |
| (95% CI) | | | | | | | | | | | | |

MCL Efficacy in Parts 1 & 2;
NE = Not estimable;
NR = Not reached;
N = number of evaluable patients;
sMIPI: Simplified Mantle Cell Lymphoma International Prognostic Index;
^{a}prior BTK inhibitor = ibrutinib;
^{b}Autologous stem cell transplant (n = 8), Allogeneic stem cell transplant (n = 1), CAR-T (n = 1) patients could have received more than one;
^{c}includes 1 unconfirmed CR.

TABLE 5
Clinical Response Rates in Heavily Pre-treated CLL Subgroups

| | Parts 1 &2 | | | | | Part 3 | | | | | Part 3 | | | | |
|---|---|---|---|---|---|---|---|---|---|---|---|---|---|---|---|
| | Zilovertamab + Ibrutinib | | | | | Zilovertamab + Ibrutinib | | | | | Ibrutinib alone | | | | |
| | | | 1 | 2 | ≥3 | | | 1 | 2 | ≥3 | | | 1 | 2 | ≥3 |
| | Overall | TN | Prior | Prior | Prior | Overall | TN | Prior | Prior | Prior | Overall | TN | Prior | Prior | Prior |
| | N = 34 | N = 12 | N = 10 | N = 4 | N = 8 | N = 16 | N = 8 | N = 4 | N = 2 | N = 2 | N = 7 | N = 4 | N = 2 | N = 0 | N = 1 |
| ORR, n | 31 | 11 | 10 | 3 | 7 | 15 | 8 | 4 | 1 | 2 | 7 | 4 | 2 | 0 | 1 |
| (%) | (91.2) | (91.7) | (100.0) | (75.0) | (87.5) | (93.8) | (100.0) | (100) | (50.0) | (100.0) | (100.0) | (100.0) | (100.0) | | (100.0) |
| CR, n | 3^{a} | 1^{a} | 2 | 0 | 0 | 0 | 0 | 0 | 0 | | | 0 | 0 | 0 | 0 |
| (%) | (8.8) | (8.3) | (20.0) | | | | | | | | | | | | |
| PR, n | 28^{b} | 10 | 8 | 3 | 6 | 15 | 8 | 4 | 1 | 2 | 7 | 4 | 2 | 0 | 1 |
| (%) | (82.3) | (83.3) | (80.0) | (75.0%) | (75.0%) | (93.8) | (100.0) | (100.0) | (100.0) | (100.0) | (100.0) | (100.0) | (100.0) | | (100.0) |
| SD, n | 3 | 1 | 0 | 1 | 1 | 1 | 0 | 0 | 1 | 0 | 0 | 0 | 0 | 0 | 0 |
| (%) | (8.8) | (8.3) | | (25.0) | (12.5) | (6.3) | | | (100.0) | | | | | | |
| PD, n | 0 | 0 | 0 | 0 | 0 | 0 | 0 | 0 | 0 | 0 | 0 | 0 | 0 | 0 | 0 |
| (%) | | | | | | | | | | | | | | | |
| Median | 33.54 | NR | NR | NR | 33.54 | NR | NR | NR | NR | NR | NR | NR | NR | NA | 8.3 |
| DoR, | (33.54, | (9.70, | (7.4, | (5.5, | (19.57, | (0, | (8.49, | (16.6. | (20.9, | (0.0, | 8.30, | (8.3, | (19.3, | | (NE) |
| months | NE) | NE) | 28.0)^{c} | 28.1)^{c} | NE) | 22.2)^{c} | NE) | 22.2)^{c} | 20.9)^{c} | 13.8)^{c} | NE) | 21.3)^{c} | 19.5)^{c} | | |
| (95% | | | | | | | | | | | | | | | |
| CI) | | | | | | | | | | | | | | | |

^{a}includes 1 unconfirmed CR;
^{b}Includes 1 PR-L
^{c}min/max used when confidence interval is not estimable;
NA = Not applicable;
NE = Not estimable;
NR = Not reached;
ORR = objective response rate;
CR = complete response;
PR = partial response;
SD = stable disease;
PD = progressive disease;
DoR = duration of response.

TABLE 6
Clinical Response Comparison Between Combination
treatment and Ibrutinib Alone

| | Zilovertamab + | Historical ibrutinib |
|---|---|---|
| Clinical | ibrutinib | Rule, 2017^{a} |
| Response | N = 28 | N = 370 |
| ORR, n (%) | 25 (89.3%) | 234 (63.2) |
| CR, n (%) | 12 (42.9%) | 74 (20.0) |
| PR, n (%) | 13 (46.4%) | 160 (45.6) |
| SD, n (%) | 1 (3.6%) | 50 (13.5) |
| PD, n (%) | 2 (7.1%) | 86 (23.2) |

MCL Efficacy in Parts 1 & 2;
^{a}Patient-level data from three single-agent ibrutinib studies, N = 370 from Rule, Br J Haematol., 2017;
b - includes 1 unconfirmed CR.

TABLE 7
Clinical Response Rates by Prior Regimen in MCL

| | | | | | Historical ibrutinib | | | |
|---|---|---|---|---|---|---|---|---|
| | Zilovertamab + ibrutinib | | | | Rule, 2017^{a} | | | |
| | | 1 | 2 | ≥3 | All | 1 | 2 | ≥3 |
| | All patients | Prior | Prior | Prior | patients | Prior | Prior | Prior |
| | N = 28 | N = 16 | N = 8 | N = 4 | N = 370 | N = 99 | N = 109 | N = 162 |
| ORR, n | 25 | 14 | 7 | 4 | 243 | 72 | 70 | 101 |
| (%) | (89.3%) | (87.5%) | (87.5%) | (100.0) | (65.7) | (72.7) | (64.2) | (62.3) |
| CR, n | 12 | 4 | 6 | 2 | 74 | 27 | 21 | 26 |
| (%) | (42.9%) | (25.0%) | (75.0%) | (50.0) | (20.0) | (27.3) | (19.3) | (16.0) |
| PR, n | 13 | 10 | 1 | 2 | 169 | 45 | 49 | 75 |
| (%) | (46.4%) | (62.5%) | (12.5%) | (50.0) | (45.7) | (45.4) | (45.0) | (46.3) |
| SD, n | 1 | 0 | 1 | 0 | 50 | 12 | 14 | 24 |
| (%) | (3.6%) | | (12.5%) | | (13.5) | (12.1) | (12.8) | (14.8) |

MCL Efficacy in Parts 1 & 2;
^{a}Patient-level data from three single-agent ibrutinib studies,
N = 370 from Rule, Br J Haematol., 2017;
b - includes 1 unconfirmed CR

TABLE 8
Clinical Response Rates by p53 Mutation in MCL

| | Zilovertamab + | | Historical ibrutinib, | |
|---|---|---|---|---|
| | ibrutinib | | Rule, 2019^{a} | |
| | p53 | No p53 | p53 | No p53 |
| | Mutation | mutation | mutation | mutation |
| | N = 7 | N = 9 | N = 20 | N = 124 |
| ORR, n (%) | 6 (85.7%) | 8 (88.9%) | 11 (55.0) | 87 (70.2) |
| CR, n (%) | 1 (14.3%) | 5 (55.6%) | 0 | 31 (25.0) |
| PR, n (%) | 5 (71.4%) | 3 (33.3%) | 11 (55.0) | 56 (45.2) |

MCL Efficacy in Parts 1 & 2; Tumor Response reflects best response achieved by each subject;
^{a}Rule, Hematologica, 2019;
b - includes 1 unconfirmed CR.

TABLE 9
Progression-Free Survival by p53 Mutation in MCL

| | Zilovertamab + | Historical Ibrutinib, |
|---|---|---|
| | ibrutinib (N = 6) | Rule 2019^{a} (N = 20) |
| p53 Status | M PFS (95% CI) | M PFS (95% CI) |
| p53 Mutation | 17.3 (2.85, NE) | 4.0 (2.1, 8.3) |
| No p53 Mutation | NR | 12.0 (7.1, 15.6) |

TABLE 10
Progression-Free Survival in CLL

| | Parts 1&2 | | | Part 3 | | |
|---|---|---|---|---|---|---|
| | Zilovertamab + | Zilovertamab + | Zilovertamab + | Zilovertamab + | | |
| | ibrutinib | ibrutinib | ibrutinib | ibrutinib | Ibrutinib | Ibrutinib |
| | Treatment | Relapsed/ | Treatment | Relapsed/ | Treatment | Relapsed/ |
| | Naïve | Refractory | Naïve | Refractory | Naïve | Refractory |
| | N = 12 | N = 22 | N = 8 | N = 8 | N = 4 | N = 3 |
| Median PFS | NR | NR | NR | NR | NR | NR |
| (95% CI) | NR | (36.26, NE) | (10.98, NE) | (12.85, 26.43) | (14.16, 30.03)^{a} | (11.05, NE) |
| Median | 33.5 | 32.5 | 24.6 | 23.8 | 24.4 | 24.7 |
| Duration of | (30.42, 37.01) | (29.75, 37.39) | (18.03, 25.93) | (16.68, 25.76) | (11.44, 34.38) | (15.79, 30.53) |
| Follow-up | | | | | | |
| in months, | | | | | | |
| (95% CI) | | | | | | |

NA = Not applicable;
NE = Not estimable;
NR = Not reached;
N = number of evaluable patients;
^{a}min/max used when confidence interval is not estimable.

TABLE 11
Progression-Free Survival in CLL by Prior Systemic Treatment

| | CLL Parts 1 & 2 |
|---|---|
| Prior Systemic Treatment | MPFS (95% CI) |
| Treatment Naïve | NR (10.10, 38.82)^{a} |
| 1 Prior Regimen | NR (19.34, 43.05)^{a} |
| 2 Prior Regimens | NR (9.05, 41.57)^{a} |
| ≥3 Prior Regimens | 36.26 (15.70, NE) |

NR = not reached;
NE = not estimable;
^{a}min, max used when confidence interval is not estimable.

TABLE 12
Landmark PFS in CLL by Prior Systemic Treatment

| | Zilovertamab + ibrutinib | | Byrd, 2019^{a} | |
|---|---|---|---|---|
| | Landmark | Landmark | Landmark | Landmark |
| Prior Systemic | PFS | PFS | PFS | PFS |
| Therapy | 24 months | 36 months | 24 months | 36 months |
| 1 or 2 | ~100% | ~100% | ~82% | ~73% |
| >2 | ~88% | ~70% | ~65% | ~50% |

^{a}PFS by prior therapies from sing-agent ibrutinib treatment in high-risk, relapsed patients with CLL (N = 195), Byrd, Blood 2019.

TABLE 13
Overall Survival in MCL & CLL Patients

| | MCL Parts | | | | | | | |
|---|---|---|---|---|---|---|---|---|
| | 1&2 | MCL Rule, | CLL Parts 1&2 | | CLL Part 3 | | | |
| | Zilovertamab + | 2017^{a} | Zilovertamab + | Zilovertamab + | Zilovertamab + | Zilovertamab + | | |
| | ibrutinib | Ibrutinib | ibrutinib | ibrutinib | ibrutinib | ibrutinib | Ibrutinib | Ibrutinib |
| | Relapsed/ | Relapsed/ | Treatment | Relapsed/ | Treatment | Relapsed/ | Treatment | Relapsed/ |
| | Refractory | Refractory | Naïve | Refractory | Naïve | Refractory | Naïve | Refractory |
| | N = 28 | N = 370 | N = 12 | N = 22 | N = 8 | N = 8 | N = 4 | N = 3 |
| Median OS | NR | 25.0 | NR | NR | NR | NR | NR | NR |
| (95% CI) | (18.85, NE) | | (25.74, 42.03)^{c} | (36.26, NE) | (12.92, 27.70)^{c} | (12.85, 26.43)^{c} | (14.16, 30.03)^{c} | (19.74, 25.05) |
| Median Duration | 15.1 | 16.8^{b} | 33.5 | 32.5 | 24.6 | 23.8 | 24.4 | 24.7 |
| of Follow up in | (14.31, 22.13) | | (30.42, 37.01) | (29.75, 37.39) | (18.03, 25.93) | (16.68, 25.76) | (11.44, 34.38) | (15.79, 30.53) |
| months, | | | | | | | | |
| (95% CI) | | | | | | | | |

OS is defined as the time from the first dose to the time of death from any cause, whichever comes first;
NE = not estimable;
NR = not reached;
^{a}Patient-level data, from three single-agent ibrutinib studies, N = 370 from Rule, Br J Haematol., 2017;
^{b}adapted median follow-up of 3 pooled trials (average = 16.8) from Rule, Br J Haematol., 2017,
^{c}min, max used when confidence interval is not estimable.

TABLE 14
CLL Efficacy by TP53 mutation/del17p: Clinical Response Rates

| | Parts 1 & 2 | Part 3 - Zilo + | Part 3 - Ibr | TP53 mutation/ |
|---|---|---|---|---|
| Endpoints | (N = 34) | Ibr (N = 16) | (N = 7) | del17p (N = 10) |
| Overall Response Rate (ORR), n (%) | 31 (91.2) | 15 (93.8) | 7 (100.0) | 10 (100.0) |
| Complete Response (CR), n (%) | 3 (8.8)^{a} | 0 | 1 (14.3) | 1 (10.0) |
| Partial Response (PR), n (%) | 28 (82.4)^{b} | 15 (93.8) | 6 (85.7) | 9(90.0)^{b} |
| Stable Disease (SD), n (%) | 3 (8.8) | 1 (6.3) | 0 | 0 |
| Median Duration of response, | 40.3 (33.5, NE) | NR (22.2, NE) | NR (8.3, NE) | 40.3 (NE, NE) |
| months (95% CI) | | | | |

N = number of evaluable patients;
NE = not estimable;
^{a}includes 1 unconfirmed CR,
^{b}includes 1 PR-L

TABLE 15
Sequences Information

| SEQ ID NO | Sequence | Name |
|---|---|---|
| 1 | GYAFTAYN | Zilovertamab HCDR1 |
| 2 | FDPYDGGS | Zilovertamab HCDR2 |
| 3 | GWYYFDY | Zilovertamab HCDR3 |
| 4 | KSISKY | Zilovertamab LCDR1 |
| 5 | SGS | Zilovertamab LCDR2 |
| 6 | QQHDESPY | Zilovertamab LCDR3 |
| 7 | QVQLQESGPGLVKPSQTLSLTCTVSGYA | Zilovertamab VH |
| | FTAYNIHWVRQAPGQGLEWMGSFDPYD | |
| | GGSSYNQKFKDRLTISKDTSKNQVVLTM | |
| | TNMDPVDTATYYCARGWYYFDYWGHG | |
| | TLVTVSS | |
| 8 | DIVMTQTPLSLPVTPGEPASISCRASKSIS | Zilovertamab VL |
| | KYLAWYQQKPGQAPRLLIYSGSTLQSGI | |
| | PPRFSGSGYGTDFTLTINNIESEDAAYYF | |
| | CQQHDESPYTFGEGTKVEIK | |
| 9 | QVQLQESGPGLVKPSQTLSLTCTVSGYA | Zilovertamab HC |
| | FTAYNIHWVRQAPGQGLEWMGSFDPYD | |
| | GGSSYNQKFKDRLTISKDTSKNQVVLTM | |
| | TNMDPVDTATYYCARGWYYFDYWGHG | |
| | TLVTVSSASTKGPSVFPLAPSSKSTSGGT | |
| | AALGCLVKDYFPEPVTVSWNSGALTSG | |
| | VHTFPAVLQSSGLYSLSSVVTVPSSSLGT | |
| | QTYICNVNHKPSNTKVDKKVEPKSCDKT | |
| | HTCPPCPAPELLGGPSVFLFPPKPKDTLM | |
| | ISRTPEVTCVVVDVSHEDPEVKFNWYVD | |
| | GVEVHNAKTKPREEQYNSTYRVVSVLT | |
| | VLHQDWLNGKEYKCKVSNKALPAPIEK | |
| | TISKAKGQPREPQVYTLPPSRDELTKNQ | |
| | VSLTCLVKGFYPSDIAVEWESNGQPENN | |
| | YKTTPPVLDSDGSFFLYSKLTVDKSRWQ | |
| | QGNVFSCSVMHEALHNHYTQKSLSLSPG | |
| | K | |
| 10 | DIVMTQTPLSLPVTPGEPASISCRASKSIS | Zilovertamab LC |
| | KYLAWYQQKPGQAPRLLIYSGSTLQSGI | |
| | PPRFSGSGYGTDFTLTINNIESEDAAYYF | |
| | CQQHDESPYTFGEGTKVEIKRTVAAPSV | |
| | FIFPPSDEQLKSGTASVVCLLNNFYPREA | |
| | KVQWKVDNALQSGNSQESVTEQDSKDS | |
| | TYSLSSTLTLSKADYEKHKVYACEVTHQ | |
| | GLSSPVTKSFNRGEC | |
| 11 | MEEPQSDPSVEPPLSQETFSDLWKLLPEN | p53 |
| | NVLSPLPSQAMDDLMLSPDDIEQWFTED | |
| | PGPDEAPRMPEAAPPVAPAPAAPTPAAP | |
| | APAPSWPLSSSVPSQKTYQGSYGFRLGF | |
| | LHSGTAKSVTCTYSPALNKMFCQLAKTC | |
| | PVQLWVDSTPPPGTRVRAMAIYKQSQH | |
| | MTEVVRRCPHHERCSDSDGLAPPQHLIR | |
| | VEGNLRVEYLDDRNTFRHSVVVPYEPPE | |
| | VGSDCTTIHYNYMCNSSCMGGMNRRPI | |
| | LTIITLEDSSGNLLGRNSFEVRVCACPGR | |
| | DRRTEEENLRKKGEPHHELPPGSTKRAL | |
| | PNNTSSSPQPKKKPLDGEYFTLQIRGRER | |
| | FEMFRELNEALELKDAQAGKEPGGSRA | |
| | HSSHLKSKKGQSTSRHKKLMFKTEGPDS | |
| | D | |
| 12 | MHRPRRRGTRPPLLALLAALLLAARGA | ROR1 |
| | AAQETELSVSAELVPTSSWNISSELNKDS | |
| | YLTLDEPMNNITTSLGQTAELHCKVSGN | |
| | PPPTIRWFKNDAPVVQEPRRLSFRSTIYG | |
| | SRLRIRNLDTTDTGYFQCVATNGKEVVS | |
| | STGVLFVKFGPPPTASPGYSDEYEEDGFC | |
| | QPYRGIACARFIGNRTVYMESLHMQGEI | |
| | ENQITAAFTMIGTSSHLSDKCSQFAIPSLC | |
| | HYAFPYCDETSSVPKPRDLCRDECEILEN | |
| | VLCQTEYIFARSNPMILMRLKLPNCEDLP | |
| | QPESPEAANCIRIGIPMADPINKNHKCYN | |
| | STGVDYRGTVSVTKSGRQCQPWNSQYP | |
| | HTHTFTALRFPELNGGHSYCRNPGNQKE | |
| | APWCFTLDENFKSDLCDIPACDSKDSKE | |
| | KNKMEILYILVPSVAIPLAIALLFFFICVC | |
| | RNNQKSSSAPVQRQPKHVRGQNVEMSM | |
| | LNAYKPKSKAKELPLSAVRFMEELGECA | |
| | FGKIYKGHLYLPGMDHAQLVAIKTLKD | |
| | YNNPQQWMEFQQEASLMAELHHPNIVC | |
| | LLGAVTQEQPVCMLFEYINQGDLHEFLI | |
| | MRSPHSDVGCSSDEDGTVKSSLDHGDFL | |
| | HIAIQIAAGMEYLSSHFFVHKDLAARNIL | |
| | IGEQLHVKISDLGLSREIYSADYYRVQSK | |
| | SLLPIRWMPPEAIMYGKFSSDSDIWSFGV | |
| | VLWEIFSFGLQPYYGFSNQEVIEMVRKR | |
| | QLLPCSEDCPPRMYSLMTECWNEIPSRR | |
| | PRFKDIHVRLRSWEGLSSHTSSTTPSGGN | |
| | ATTQTTSLSASPVSNLSNPRYPNYMFPSQ | |
| | GITPQGQIAGFIGPPIPQNQRFIPINGYPIPP | |
| | GYAAFPAAHYQPTGPPRVIQHCPPPKSRS | |
| | PSSASGSTSTGHVTSLPSSGSNQEANIPLL | |
| | PHMSIPNHPGGMGITVFGNKSQKPYKID | |
| | SKQASLLGDANIHGHTESMISAEL | |
| 13 | VATNGKEVVSSTGVLFVKFGP | partial ROR1 |
| 14 | EVVSSTGVLFVKFGP | partial ROR1 |
| 15 | GFSLTSYG | D10 HCDR1 |
| 16 | IWAGGFT | D10 HCDR2 |
| 17 | RGSSYSMDY | D10 HCDR3 |
| 18 | SNVSY | D10 LCDR1 |
| 19 | EIS | D10 LCDR2 |
| 20 | QQWNYPLIT | D10 LCDR3 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a cancer or tumor in an individual, comprising administering to the individual an ROR1 antagonist and/or a Bruton's tyrosine kinase inhibitor (BTKi), wherein the tumor or cancer comprises a mutation in a TP53 gene or a high-risk subtype.

2. The method of claim 1, wherein the ROR1 antagonist is an antibody or antigen binding fragment thereof.

3. The method of claim 1 or claim 2, wherein the antibody or antigen binding fragment thereof binds to an epitope of ROR1 as set forth in SEQ ID NO: 13 or 14.

4. The method of claim 2 or 3, wherein the antibody or antigen biding fragment thereof comprises:
a) a heavy chain CDR1 (HCDR1) comprising the amino acid sequence set forth in SEQ ID NO: 1;
b) a heavy chain CDR2 (HCDR2) comprising the amino acid sequence set forth in SEQ ID NO: 2;
c) a heavy chain CDR3 (HCDR3) comprising the amino acid sequence set forth in SEQ ID NO: 3;
d) a light chain CDR1 (LCDR1) comprising the amino acid sequence set forth in SEQ ID NO: 4;
e) a light chain CDR2 (LCDR2) comprising the amino acid sequence set forth in SEQ ID NO: 5; and/or
f) a light chain CDR3 (LCDR3) comprising the amino acid sequence set forth in SEQ ID NO: 6.

5. The method of claim 2 or 3, wherein the antibody or antigen biding fragment thereof comprises a heavy chain variable region comprising an amino acid sequence at least about 85% identical to that set forth in SEQ ID NO: 7; and/or a light chain variable region comprising an amino acid sequence at least about 85% identical to that set forth in SEQ ID NO: 8.

6. The method of claim 2 or 3, wherein the antibody or antigen biding fragment thereof is zilovertamab or an ROR1 binding fragment thereof.

7. The method of any one of claims 1 to 6, wherein the ROR1 antagonist is administered at a dose of between 200 milligrams and 800 milligrams.

8. The method of any one of claims 1 to 6, wherein the ROR1 antagonist is administered at a dose of 200 milligrams.

9. The method of any one of claims 1 to 6, wherein the ROR1 antagonist is administered at a dose of 400 milligrams.

10. The method of any one of claims 1 to 6, wherein the ROR1 antagonist is administered at a dose of 600 milligrams.

11. The method of any one of claims 1 to 10, wherein the BTKi comprises ibrutinib, acalabrutinib, evobrutinib, fenebrutinib, orelabrutinib, rilzabrutinib, or tolebrutinib.

12. The method of any one of claims 1 to 10, wherein the BTKi comprises branebrutinib, elsubrutinib, remibrutinib, vecabrutinib, remibrutinib, spebrutinib tirabrutinib, AC0058TA, pirtobrutinib (LOXO-305), DTRMWXHS-12, M7583, SN-1011, TG-1701.

13. The method of any one of claims 1 to 10, wherein the BTKi comprises ibrutinib.

14. The method of any one of claims 1 to 10, wherein the BTKi comprises nemtabrutinib.

15. The method of any one of claims 1 to 14, wherein the BTKi is administered at a dose of between about 280 and about 700 milligrams.

16. The method of any one of claims 1 to 14, wherein the BTKi is administered at a dose of about 420 milligrams.

17. The method of any one of claims 1 to 14, wherein the BTKi is administered at a dose of about 560 milligrams.

18. The method of any one of claims 1 to 17, wherein the ROR1 antagonist and the BTKi are administered separately.

19. The method of any one of claims 1 to 18, wherein the tumor or the cancer comprises a blood cancer.

20. The method of any one of claims 1 to 18, wherein the tumor or the cancer comprises a leukemia or a lymphoma.

21. The method of any one of claims 1 to 18, wherein the tumor or the cancer comprises a B cell leukemia or B cell lymphoma.

22. The method of any one of claims 1 to 18, wherein the tumor or the cancer comprises chronic lymphocytic leukemia (CLL).

23. The method of any one of claims 1 to 18, wherein the tumor or the cancer comprises small cell lymphocytic leukemia (SLL).

24. The method of any one of claims 1 to 18, wherein the tumor or the cancer comprises marginal zone lymphoma (MZL).

25. The method of any one of claims 1 to 18, wherein the tumor or the cancer comprises diffuse large B cell lymphoma (DLBCL).

26. The method of any one of claims 1 to 18, wherein the tumor or the cancer comprises mantle cell lymphoma (MCL).

27. The method of any one of claims 1 to 18, wherein the MCL is a high-risk subtype according to the simplified mantle cell lymphoma international prognostic index.

28. The method of any one of claims 1 to 27, wherein the mutation is in an exon of the TP53 gene.

29. The method of any one of claims 1 to 27, wherein the mutation is in an intron of the TP53 gene.

30. The method of any one of claims 1 to 27, wherein the mutation is a nonsense mutation in the TP53 gene.

31. The method of any one of claims 1 to 27, wherein the mutation is a missense mutation in the TP53 gene.

32. The method of any one of claims 1 to 27, wherein the mutation results in an amino acid insertion, deletion, or substitution in a protein encoded by the TP53 gene.

33. The method of any one of claims 1 to 32, wherein the cancer or tumor is relapsed.

34. The method of any one of claims 1 to 32, wherein the cancer or tumor is refractory to one or more previous treatments.

35. The method of any one of claims 1 to 34, wherein the test is performed on a biopsy sample of the tumor or the cancer of the individual.


SEQUENCE LISTING (20 sequences)

SEQ ID NO: 1-6 - skipped (6 entries)

SEQ ID NO: 7 - protein, 116 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 8 - protein, 107 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 9 - protein, 446 aa, synthetic construct
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 393 aa, synthetic construct [sequence omitted]

SEQ ID NO: 12 - protein, 937 aa, synthetic construct
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWMEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 13 - protein, 21 aa, synthetic construct
VATNGKEVVSSTGVLFVKFGP

SEQ ID NO: 14 - protein, 15 aa, synthetic construct
EVVSSTGVLFVKFGP

SEQ ID NO: 15-20 - skipped (6 entries)